SEQUENCE LISTING (14 sequences)

SEQ ID NO: 1 - DNA, 3810 bp, synthetic construct
  source 1..3810: mol_type = other DNA
atgctgtggtgggaggaagtggaagattgctacgagcgcgaggacgtgcagaagaaaaccttcaccaaatgggtcaacgcccagttcagcaagttcggcaagcagcacatcgagaacctgttcagcgacctgcaggacggcagacggctgctggatctgctggaaggcctgaccggacagaagctgcccaaagagaagggcagcaccagagtgcacgccctgaacaacgtgaacaaggccctgcgggtgctgcagaacaacaatgtggacctggtgaacattggcagcacagacattgtggatggcaaccacaagctgaccctgggcctgatctggaacatcatcctgcactggcaagtgaagaacgtgatgaagaacatcatggccggcctgcagcagaccaacagcgagaagatcctgctgagctgggtgcgccagagcaccagaaactacccccaagtgaacgtgatcaacttcaccacctcttggagcgacggcctggccctgaatgccctgatccacagccacagacctgacctgtttgactggaacagtgttgtgtgtcagcagagtgccacccagaggctggaacacgccttcaatatcgccagataccagctgggcatcgagaagctgctggaccccgaggatgtggacaccacctaccccgacaagaaatccatcctgatgtatatcaccagcctgttccaggtgctgcctcagcaggtgtccatcgaggccatccaggaagtggaaatgctgcccagaccccccaaagtgaccaaagaggaacacttccagctgcaccaccagatgcactactctcagcagatcaccgtgtccctggcccagggctacgagagaaccagcagccccaagccccggttcaagagctacgcctatacccaggccgcctacgtgaccaccagcgaccctaccagaagcccattccccagccagcatctggaagcccccgaggacaagagcttcggcagcagcctgatggaaagcgaagtgaacctggatagataccagaccgccctggaagaggtgctgtcctggctgctgagcgccgaggatacactgcaggctcagggcgagatcagcaacgacgtggaagtcgtgaaggaccagttccacacccacgagggctacatgatggacctgacagcccaccagggcagagtgggcaacattctgcagctgggctccaagctgatcggcaccggcaagctgagcgaggacgaagagacagaggtgcaggaacagatgaacctgctgaacagcagatgggagtgcctgagagtggccagcatggaaaagcagagcaacctgcacagctacgtgcccagcacctacctgaccgagatcacccatgtgtcccaggccctgctggaagtggaacagctgctgaacgcccccgatctgtgcgccaaggacttcgaggatctgttcaagcaggaagagagcctgaagaatatcaaggactctctgcagcagtccagcggcagaatcgacatcatccacagcaagaaaacagccgccctgcagtccgccacccccgtggaaagagtgaagctgcaggaagccctgtcccagctggacttccagtgggagaaagtgaacaagatgtacaaggacaggcagggcagatttgacaggtctgtggaaaagtggaggaggttccactacgacatcaagatcttcaaccagtggctgaccgaggccgagcagttcctgagaaagacccagatccccgagaactgggagcacgccaagtacaagtggtatctgaaagaactgcaggatggcatcggccagagacagacagttgtgaggacactgaatgccacaggagaggaaatcatccagcagagcagcaagacagatgccagtattctgcaggaaaagctgggcagcctgaacctgagatggcaggaagtgtgcaagcagctgtccgaccggaagaagagactggaagaacagagcgaccagtggaagcggctgcatctgtcactgcaggaactgctcgtgtggctgcagctgaaggatgatgagctgagcagacaggcccctattggaggagattttcctgctgtgcagaaacagaatgatgtgcaccgggccttcaagagagagctgaaaacaaaagaacccgtgatcatgagcaccctggaaaccgtgcggatctttctgaccgagcagcccctggaaggactggaaaaactgtaccaggaacccagagagctgccccctgaagaacgggcccagaacgtgaccagactgctgcggaagcaggccgaggaagtgaacaccgaatgggagaagctgaacctgcactctgctgactggcagaggaagattgatgagacactggaacggctgcaggaactgcaggaggccaccgacgagctggacctgaaactgagacaggccgaagtgatcaagggcagctggcagccagtgggcgacctgctgatcgacagcctgcaggatcacctggaaaaagtgaaagccctgagaggcgagatcgcccccctgaaagaaaacgtgtcccacgtgaacgacctggcccggcagctgacaacactgggcatccagctgagcccctacaacctgtccacactggaagatctgaacacccggtggaaactgctgcaggtggccgtggaagatagagtgcggcagctgcacgaggcccacagagattttggccctgcctcccagcacttcctgagcacatctgtgcagggcccctgggagagagccatctcccccaacaaggtgccctactacatcaaccacgagacacagaccacctgttgggaccaccccaagatgaccgagctgtaccagagcctggccgacctgaacaatgtgaggttcagtgcctacaggacagccatgaagctgaggagactgcagaaagctctgtgcctggacctgctgtccctgtccgccgcttgtgatgccctggaccagcacaacctgaagcagaacgaccagcccatggatatcctgcagatcatcaactgcctgaccaccatctacgaccgcctggaacaggaacacaacaacctcgtgaatgtgcccctgtgcgtggacatgtgcctgaattggctgctgaatgtgtatgacacaggcaggacaggcaggatcagagtgctgagcttcaagaccggcatcatcagcctgtgcaaggcccacctggaagataagtaccgctacctgttcaaacaggtggccagctccaccggcttttgcgaccagagaaggctgggcctgctgctgcacgacagcatccagatccctagacagctgggcgaggtggcctcttttggaggcagcaatattgagcctagtgtgaggagctgctttcagttcgccaacaacaagcctgagattgaggctgccctgttcctggactggatgcggctggaaccccagagcatggtgtggctgcctgtgctgcatagagtggccgctgccgagacagccaagcaccaggccaagtgcaacatctgcaaagagtgccccatcatcggcttccggtacagaagcctgaagcacttcaactacgatatctgccagagctgctttttcagcggacgggtggccaagggccacaaaatgcactaccccatggtggaatactgcacccccaccacctccggggaggatgtgcgggattttgccaaggtgctgaaaaacaagttccggaccaagcgctacttcgccaaacacccccggatgggctatctgcccgtgcagacagtgctggaaggcgacaacatggaaaccgacaccatg

SEQ ID NO: 2 - DNA, 3813 bp, synthetic construct
  source 1..3813: mol_type = other DNA
atgctgtggtgggaggaagtggaagattgctacgagcgcgaggacgtgcagaagaaaaccttcaccaaatgggtcaacgcccagttcagcaagttcggcaagcagcacatcgagaacctgttcagcgacctgcaggacggcagacggctgctggatctgctggaaggcctgaccggacagaagctgcccaaagagaagggcagcaccagagtgcacgccctgaacaacgtgaacaaggccctgcgggtgctgcagaacaacaatgtggacctggtgaacattggcagcacagacattgtggatggcaaccacaagctgaccctgggcctgatctggaacatcatcctgcactggcaagtgaagaacgtgatgaagaacatcatggccggcctgcagcagaccaacagcgagaagatcctgctgagctgggtgcgccagagcaccagaaactacccccaagtgaacgtgatcaacttcaccacctcttggagcgacggcctggccctgaatgccctgatccacagccacagacctgacctgtttgactggaacagtgttgtgtgtcagcagagtgccacccagaggctggaacacgccttcaatatcgccagataccagctgggcatcgagaagctgctggaccccgaggatgtggacaccacctaccccgacaagaaatccatcctgatgtatatcaccagcctgttccaggtgctgcctcagcaggtgtccatcgaggccatccaggaagtggaaatgctgcccagaccccccaaagtgaccaaagaggaacacttccagctgcaccaccagatgcactactctcagcagatcaccgtgtccctggcccagggctacgagagaaccagcagccccaagccccggttcaagagctacgcctatacccaggccgcctacgtgaccaccagcgaccctaccagaagcccattccccagccagcatctggaagcccccgaggacaagagcttcggcagcagcctgatggaaagcgaagtgaacctggatagataccagaccgccctggaagaggtgctgtcctggctgctgagcgccgaggatacactgcaggctcagggcgagatcagcaacgacgtggaagtcgtgaaggaccagttccacacccacgagggctacatgatggacctgacagcccaccagggcagagtgggcaacattctgcagctgggctccaagctgatcggcaccggcaagctgagcgaggacgaagagacagaggtgcaggaacagatgaacctgctgaacagcagatgggagtgcctgagagtggccagcatggaaaagcagagcaacctgcacagctacgtgcccagcacctacctgaccgagatcacccatgtgtcccaggccctgctggaagtggaacagctgctgaacgcccccgatctgtgcgccaaggacttcgaggatctgttcaagcaggaagagagcctgaagaatatcaaggactctctgcagcagtccagcggcagaatcgacatcatccacagcaagaaaacagccgccctgcagtccgccacccccgtggaaagagtgaagctgcaggaagccctgtcccagctggacttccagtgggagaaagtgaacaagatgtacaaggacaggcagggcagatttgacaggtctgtggaaaagtggaggaggttccactacgacatcaagatcttcaaccagtggctgaccgaggccgagcagttcctgagaaagacccagatccccgagaactgggagcacgccaagtacaagtggtatctgaaagaactgcaggatggcatcggccagagacagacagttgtgaggacactgaatgccacaggagaggaaatcatccagcagagcagcaagacagatgccagtattctgcaggaaaagctgggcagcctgaacctgagatggcaggaagtgtgcaagcagctgtccgaccggaagaagagactggaagaacagagcgaccagtggaagcggctgcatctgtcactgcaggaactgctcgtgtggctgcagctgaaggatgatgagctgagcagacaggcccctattggaggagattttcctgctgtgcagaaacagaatgatgtgcaccgggccttcaagagagagctgaaaacaaaagaacccgtgatcatgagcaccctggaaaccgtgcggatctttctgaccgagcagcccctggaaggactggaaaaactgtaccaggaacccagagagctgccccctgaagaacgggcccagaacgtgaccagactgctgcggaagcaggccgaggaagtgaacaccgaatgggagaagctgaacctgcactctgctgactggcagaggaagattgatgagacactggaacggctgcaggaactgcaggaggccaccgacgagctggacctgaaactgagacaggccgaagtgatcaagggcagctggcagccagtgggcgacctgctgatcgacagcctgcaggatcacctggaaaaagtgaaagccctgagaggcgagatcgcccccctgaaagaaaacgtgtcccacgtgaacgacctggcccggcagctgacaacactgggcatccagctgagcccctacaacctgtccacactggaagatctgaacacccggtggaaactgctgcaggtggccgtggaagatagagtgcggcagctgcacgaggcccacagagattttggccctgcctcccagcacttcctgagcacatctgtgcagggcccctgggagagagccatctcccccaacaaggtgccctactacatcaaccacgagacacagaccacctgttgggaccaccccaagatgaccgagctgtaccagagcctggccgacctgaacaatgtgaggttcagtgcctacaggacagccatgaagctgaggagactgcagaaagctctgtgcctggacctgctgtccctgtccgccgcttgtgatgccctggaccagcacaacctgaagcagaacgaccagcccatggatatcctgcagatcatcaactgcctgaccaccatctacgaccgcctggaacaggaacacaacaacctcgtgaatgtgcccctgtgcgtggacatgtgcctgaattggctgctgaatgtgtatgacacaggcaggacaggcaggatcagagtgctgagcttcaagaccggcatcatcagcctgtgcaaggcccacctggaagataagtaccgctacctgttcaaacaggtggccagctccaccggcttttgcgaccagagaaggctgggcctgctgctgcacgacagcatccagatccctagacagctgggcgaggtggcctcttttggaggcagcaatattgagcctagtgtgaggagctgctttcagttcgccaacaacaagcctgagattgaggctgccctgttcctggactggatgcggctggaaccccagagcatggtgtggctgcctgtgctgcatagagtggccgctgccgagacagccaagcaccaggccaagtgcaacatctgcaaagagtgccccatcatcggcttccggtacagaagcctgaagcacttcaactacgatatctgccagagctgctttttcagcggacgggtggccaagggccacaaaatgcactaccccatggtggaatactgcacccccaccacctccggggaggatgtgcgggattttgccaaggtgctgaaaaacaagttccggaccaagcgctacttcgccaaacacccccggatgggctatctgcccgtgcagacagtgctggaaggcgacaacatggaaaccgacaccatgtag

SEQ ID NO: 3 - DNA, 4784 bp, synthetic construct
  source 1..4784: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatccaagctttagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaaaactagtgtatcaaggttacaagacaggtttaaggagaccaatagaaactgggcttgtcgagacagagaagactcttgcgtttctgataggcacctattggtcttactgacatccactttgcctttctctccacaggcctctagaaccatgctgtggtgggaggaagtggaagattgctacgagcgcgaggacgtgcagaagaaaaccttcaccaaatgggtcaacgcccagttcagcaagttcggcaagcagcacatcgagaacctgttcagcgacctgcaggacggcagacggctgctggatctgctggaaggcctgaccggacagaagctgcccaaagagaagggcagcaccagagtgcacgccctgaacaacgtgaacaaggccctgcgggtgctgcagaacaacaatgtggacctggtgaacattggcagcacagacattgtggatggcaaccacaagctgaccctgggcctgatctggaacatcatcctgcactggcaagtgaagaacgtgatgaagaacatcatggccggcctgcagcagaccaacagcgagaagatcctgctgagctgggtgcgccagagcaccagaaactacccccaagtgaacgtgatcaacttcaccacctcttggagcgacggcctggccctgaatgccctgatccacagccacagacctgacctgtttgactggaacagtgttgtgtgtcagcagagtgccacccagaggctggaacacgccttcaatatcgccagataccagctgggcatcgagaagctgctggaccccgaggatgtggacaccacctaccccgacaagaaatccatcctgatgtatatcaccagcctgttccaggtgctgcctcagcaggtgtccatcgaggccatccaggaagtggaaatgctgcccagaccccccaaagtgaccaaagaggaacacttccagctgcaccaccagatgcactactctcagcagatcaccgtgtccctggcccagggctacgagagaaccagcagccccaagccccggttcaagagctacgcctatacccaggccgcctacgtgaccaccagcgaccctaccagaagcccattccccagccagcatctggaagcccccgaggacaagagcttcggcagcagcctgatggaaagcgaagtgaacctggatagataccagaccgccctggaagaggtgctgtcctggctgctgagcgccgaggatacactgcaggctcagggcgagatcagcaacgacgtggaagtcgtgaaggaccagttccacacccacgagggctacatgatggacctgacagcccaccagggcagagtgggcaacattctgcagctgggctccaagctgatcggcaccggcaagctgagcgaggacgaagagacagaggtgcaggaacagatgaacctgctgaacagcagatgggagtgcctgagagtggccagcatggaaaagcagagcaacctgcacagctacgtgcccagcacctacctgaccgagatcacccatgtgtcccaggccctgctggaagtggaacagctgctgaacgcccccgatctgtgcgccaaggacttcgaggatctgttcaagcaggaagagagcctgaagaatatcaaggactctctgcagcagtccagcggcagaatcgacatcatccacagcaagaaaacagccgccctgcagtccgccacccccgtggaaagagtgaagctgcaggaagccctgtcccagctggacttccagtgggagaaagtgaacaagatgtacaaggacaggcagggcagatttgacaggtctgtggaaaagtggaggaggttccactacgacatcaagatcttcaaccagtggctgaccgaggccgagcagttcctgagaaagacccagatccccgagaactgggagcacgccaagtacaagtggtatctgaaagaactgcaggatggcatcggccagagacagacagttgtgaggacactgaatgccacaggagaggaaatcatccagcagagcagcaagacagatgccagtattctgcaggaaaagctgggcagcctgaacctgagatggcaggaagtgtgcaagcagctgtccgaccggaagaagagactggaagaacagagcgaccagtggaagcggctgcatctgtcactgcaggaactgctcgtgtggctgcagctgaaggatgatgagctgagcagacaggcccctattggaggagattttcctgctgtgcagaaacagaatgatgtgcaccgggccttcaagagagagctgaaaacaaaagaacccgtgatcatgagcaccctggaaaccgtgcggatctttctgaccgagcagcccctggaaggactggaaaaactgtaccaggaacccagagagctgccccctgaagaacgggcccagaacgtgaccagactgctgcggaagcaggccgaggaagtgaacaccgaatgggagaagctgaacctgcactctgctgactggcagaggaagattgatgagacactggaacggctgcaggaactgcaggaggccaccgacgagctggacctgaaactgagacaggccgaagtgatcaagggcagctggcagccagtgggcgacctgctgatcgacagcctgcaggatcacctggaaaaagtgaaagccctgagaggcgagatcgcccccctgaaagaaaacgtgtcccacgtgaacgacctggcccggcagctgacaacactgggcatccagctgagcccctacaacctgtccacactggaagatctgaacacccggtggaaactgctgcaggtggccgtggaagatagagtgcggcagctgcacgaggcccacagagattttggccctgcctcccagcacttcctgagcacatctgtgcagggcccctgggagagagccatctcccccaacaaggtgccctactacatcaaccacgagacacagaccacctgttgggaccaccccaagatgaccgagctgtaccagagcctggccgacctgaacaatgtgaggttcagtgcctacaggacagccatgaagctgaggagactgcagaaagctctgtgcctggacctgctgtccctgtccgccgcttgtgatgccctggaccagcacaacctgaagcagaacgaccagcccatggatatcctgcagatcatcaactgcctgaccaccatctacgaccgcctggaacaggaacacaacaacctcgtgaatgtgcccctgtgcgtggacatgtgcctgaattggctgctgaatgtgtatgacacaggcaggacaggcaggatcagagtgctgagcttcaagaccggcatcatcagcctgtgcaaggcccacctggaagataagtaccgctacctgttcaaacaggtggccagctccaccggcttttgcgaccagagaaggctgggcctgctgctgcacgacagcatccagatccctagacagctgggcgaggtggcctcttttggaggcagcaatattgagcctagtgtgaggagctgctttcagttcgccaacaacaagcctgagattgaggctgccctgttcctggactggatgcggctggaaccccagagcatggtgtggctgcctgtgctgcatagagtggccgctgccgagacagccaagcaccaggccaagtgcaacatctgcaaagagtgccccatcatcggcttccggtacagaagcctgaagcacttcaactacgatatctgccagagctgctttttcagcggacgggtggccaagggccacaaaatgcactaccccatggtggaatactgcacccccaccacctccggggaggatgtgcgggattttgccaaggtgctgaaaaacaagttccggaccaagcgctacttcgccaaacacccccggatgggctatctgcccgtgcagacagtgctggaaggcgacaacatggaaaccgacaccatgtaggaagtcttttaataaaagatccttattttcattggatctgtgtgttggttttttgtgtgctcgagggatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 4 - DNA, 145 bp, Adeno-associated virus
  source 1..145: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 5 - DNA, 145 bp, Adeno-associated virus
  source 1..145: mol_type = genomic DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 6 - DNA, 447 bp, synthetic construct
  source 1..447: mol_type = other DNA
tagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagcca

SEQ ID NO: 7 - DNA, 810 bp, synthetic construct
  source 1..810: mol_type = other DNA
gtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcggcgcgccc

SEQ ID NO: 8 - DNA, 3810 bp, Homo sapiens
  source 1..3810: mol_type = genomic DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacattcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaatctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatg

SEQ ID NO: 9 - DNA, 48 bp, synthetic construct
  source 1..48: mol_type = other DNA
aataaaagatccttattttcattggatctgtgtgttggttttttgtgt

SEQ ID NO: 10 - protein, 1270 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHSYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 11 - DNA, 2217 bp, Adeno-associated virus
  source 1..2217: mol_type = genomic DNA
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcccgctaaaaagagactgaactttggtcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatatccaatgggacatcgggaggaagcaccaacgacaacacctacttcggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggacgtcttcatgattccccagtacggctaccttacactgaacaatggaagtcaagccgtaggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcatcgaccagtacctgtactacttatccagaactcagtccacaggaggaactcaaggtacccagcaattgttattttctcaagctgggcctgcaaacatgtcggctcaggctaagaactggctacctggaccttgctaccggcagcagcgagtctctacgacactgtcgcaaaacaacaacagcaactttgcttggactggtgccaccaaatatcacctgaacggaagagactctttggtaaatcccggtgtcgccatggcaacccacaaggacgacgaggaacgcttcttcccgtcgagtggagtcctgatgttcggaaaacagggtgctggaagagacaatgtggactacagcagcgttatgctaaccagcgaagaagaaattaaaaccactaaccccgtagccacagaacaatacggtgtggtggctgacaacttgcagcaaaccaatacagggcctattgtgggaaatgtcaacagccaaggagccttacctggcatggtctggcagaaccgagacgtgtacctgcagggtcccatctgggccaagattcctcacacggacggcaacttccacccttcaccgctaatgggaggatttggactgaagcacccacctcctcagatcctgatcaagaacacgccggtacctgcggatcctccaacaacgttcagccaggcgaaattggcttccttcattacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttacctcacccgtaatctgtaa

SEQ ID NO: 12 - protein, 738 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 13 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 14 - protein, 349 aa, Streptococcus equi
MKTIAYPNKPHSLSAGLLTAIAIFSLASSNITYADDYQRNATEAYAKEVPHQITSVWSKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

(19) United States
(12) Patent Application Publication
(10) Pub. No.: US 2025/0019721 A1
(43) Pub. Date: Jan. 16, 2025

(54) COMPOSITIONS AND METHODS FOR TREATING A MUSCULAR DYSTROPHY

(71) Applicant: Ultragenyx Pharmaceutical Inc., Novato, CA (US)

(72) Inventors: Matthew Scott Fuller, Millis, MA (US); Lorelei Ioana Stoica, Acton, MA (US); Aubrey Rose Tiernan, Somerville, MA (US); Kelly Reed Clark, Westerville, OH (US); Samuel Wadsworth, Monterey, MA (US)

(21) Appl. No.: 18/682,225

(22) PCT Filed: Aug. 10, 2022
(86) PCT No.: PCT/US2022/074760

§ 371 Date: Feb. 8, 2024

Related U.S. Application Data
(60) Provisional application No. 63/231,752, filed on Aug. 11, 2021.

(51) Int. Cl. C12N 15/86

(52) U.S. Cl. CPC C12N 15/86; C12N 2750/14143; C12N 2830/008; C12N 2830/50

(57) ABSTRACT

The present disclosure provides novel synthetic nucleic acids and recombinant adeno-associated virus (rAAV) comprising the same, as well as methods of their use in the treatment of muscular dystrophies associated with a dystrophin mutation. Also provided are pharmaceutical compositions comprising a novel synthetic nucleic acid or rAAV of the invention, and a pharmaceutically acceptable carrier or excipient. Pharmaceutical compositions comprising an rAAV of the invention may be useful in gene therapy for the treatment of dystrophin-associated muscular dystrophies, such as Duchenne muscular dystrophy (DVD). Becker muscular dystrophy (BMD), and X-linked cardiomyopathy.

### CROSS-REFERENCE TO RELATED APPLICATION

This application claims the benefit of and priority to U.S. Provisional Application No. 63/231,752, filed on Aug. 11, 2021, the entire disclosure of which is hereby incorporated by reference herein in its entirety for all purposes.

### REFERENCE TO A SEQUENCE LISTING XML

This application contains a Sequence Listing which has been submitted electronically in XML format. The Sequence Listing XML is incorporated herein by reference. Said XML file, created on Aug. 3, 2022, is named ULP-011WO.xml and is 35,812 bytes in size.

### TECHNICAL FIELD OF THE INVENTION

The present disclosure relates generally to novel nucleic acids encoding micro-dystrophin, recombinant adeno-associated viral vectors, recombinant adeno-associated virus, and methods of their use in gene therapy for treating one or more muscular dystrophies.

### BACKGROUND OF THE INVENTION

Muscular dystrophies are a group of monogenic inherited muscle disorders characterized by progressive muscle wasting and weakness. The first gene associated with muscular dystrophy, dystrophin, was cloned by Kunkel et al. in 1987. See Koenig et al., 1987. Cell 50 (3): 509-17 and Kunkel, 2005, Am. J. Hum. Genet. 76:205-14. Mutations in dystrophin are responsible for various forms of muscular dystrophy, including Duchenne muscular dystrophy (DMD), Becker muscular dystrophy, and X-linked dilated cardiomyopathy. DMD is a genetic muscle wasting disorder that affects approximately one in 3500 males. DMD patients generally carry at least one mutation in the dystrophin gene that causes aberrant expression or loss of expression of the dystrophin protein. Patients with DMD experience progressive wasting of skeletal muscles and cardiac dysfunction, which leads to loss of ambulation and premature death, primarily resulting from cardiac or respiratory failure. Unfortunately, currently available treatments are generally only able to slow the progression of DMD. Accordingly, there is an urgent need for improved compositions and methods for treating DMD and other disorders associated with dystrophin mutation.

### SUMMARY OF THE INVENTION

This invention provides compositions and methods of their use in gene therapy. More specifically, provided herein are synthetic nucleic acids encoding a micro-dystrophin protein. Also provided are recombinant adeno-associated virus (rAAV) comprising an adeno-associated virus (AAV) capsid and a vector genome packaged therein (i.e., a “packaged vector genome”) which comprises a synthetic nucleic acid encoding a micro-dystrophin protein. The synthetic nucleic acids and rAAV described herein may be used in methods for the treatment of muscular dystrophies such as DMD.

In one aspect, the present disclosure provides novel synthetic nucleic acid sequences encoding a micro-dystrophin protein. In one embodiment, the present disclosure provides a nucleic acid encoding a micro-dystrophin protein, wherein the nucleic acid comprises a sequence that is at least 80%, at least 85%, at least 90%, at least 91%, at least 92%, at least 93%, at least 94%, at least 95%, at least 96%, at least 97%, at least 98%, at least 99%, at least 99.5%, at least 99.9% or more identical to SEQ ID NO: 1. In an exemplary embodiment, the present disclosure provides a nucleic acid encoding a micro-dystrophin protein, wherein the nucleic acid comprises a sequence that is at least 99% (e.g., from 99% to 100%) identical to SEQ ID NO: 1. In a further exemplary embodiment, the present disclosure provides a nucleic acid encoding a micro-dystrophin protein, wherein the nucleic acid comprises or consists of the sequence set forth in SEQ ID NO: 1. Further provided are fragments of the nucleic acid sequence shown in SEQ ID NO: 1 which encode a polypeptide having functional micro-dystrophin activity. In some embodiments, the nucleic acid sequence encoding a micro-dystrophin protein, e.g., SEQ ID NO: 1 may further comprise a stop codon (TGA, TAA, or TAG) at the 3′ end, e.g., such as the sequence exemplified set forth in SEQ ID NO: 2 that includes a TAG stop codon at the 3″ end of SEQ ID NO: 1.

In another aspect, the present disclosure provides novel vector genome constructs useful in the treatment of a muscular dystrophy, e.g., DMD, Becker muscular dystrophy, or X-linked dilated cardiomyopathy. In one embodiment, the present disclosure provides a vector genome construct (i.e., a polynucleotide) encoding a micro-dystrophin protein, wherein the vector genome construct (i.e., a polynucleotide) is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 3. In an exemplary embodiment, the present disclosure provides a polynucleotide encoding a micro-dystrophin protein, wherein the polynucleotide is at least 99% (e.g., from 99% to 100%) identical to SEQ ID NO: 3. In a further exemplary embodiment, the present disclosure provides a polynucleotide encoding a micro-dystrophin protein, wherein the polynucleotide comprises or consists of the sequence set forth in SEQ ID NO: 3.

In yet another aspect, the present disclosure provides a recombinant adeno-associated virus (rAAV) comprising an AAV capsid, and a vector genome packaged therein, wherein said vector genome comprises a nucleic acid encoding a micro-dystrophin protein, and wherein the nucleic acid is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 1. In an exemplary embodiment, the nucleic acid comprises a sequence that is at least 99% (e.g., from 99% to 100%) identical to SEQ ID NO: 1. In a further exemplary embodiment, the nucleic acid comprises or consists of the sequence set forth in SEQ ID NO: 1.

In some embodiments, the packaged vector genome may further comprise one or more additional sequence elements selected from: (a) a 5′-inverted terminal repeat (ITR); (b) a muscle specific control element; (c) an intron; (d) a polyadenylation signal sequence; and (e) a 3-inverted terminal repeat (ITR). In some embodiments, the packaged vector genome is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 3. In an exemplary embodiment, the packaged vector genome is at least 99% (e.g., from 99% to 100%) identical to SEQ ID NO: 3. In a further exemplary embodiment, the packaged vector genome comprises or consists of the sequence set forth in SEQ ID NO: 3.

In embodiments where the packaged vector genome comprises a 5′-ITR, the 5′-ITR may be from AAV2. In some embodiments, the 5′-ITR comprises or consists of SEQ ID NO: 4 (in the plus/plus strand orientation). In other embodiments, the 5′-ITR is from a non-AAV2 source.

In embodiments where the packaged vector genome comprises a 3′-ITR, the 3-ITR may be from AAV2. In some embodiments, the 3″-ITR comprises or consists of SEQ ID NO: 4 (in the plus/minus strand orientation), which corresponds to SEQ ID NO: 5 in the plus/plus strand orientation. In other embodiments, the 3′-ITR is from a non-AAV2 source.

In embodiments where the packaged vector genome comprises a muscle specific control element (e.g., an enhancer and/or a promoter), the muscle specific control element may be selected from a CK8 promoter, a CK7 promoter, a CK9 promoter, a muscle specific creatine kinase (MCK) promoter, truncated MCK (tMCK), myosin heavy chain (MHC), hybrid α-myosin heavy chain enhancer-/MCK (MHCK7) enhancer-promoter, a human skeletal actin gene element, a cardiac actin gene element, a myocyte-specific enhancer binding factor mef, C5-12, a murine creatine kinase enhancer element, a skeletal fast-twitch troponin c gene element, a slow-twitch cardiac troponin c gene element, the slow-twitch troponin i gene element, a hypoxia-inducible nuclear factor, a steroid-inducible element, and a glucocorticoid response element (gre). In an exemplary embodiment, the muscle specific control element is selected from a CK8 promoter and a hybrid α-myosin heavy chain enhancer-/MCK (MHCK7) enhancer-promoter. In a further exemplary embodiment, the muscle specific control element is a CK8 promoter comprising or consisting of a sequence set forth in SEQ ID NO: 6. In some embodiments, the muscle specific control element comprises a sequence that is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 6. In an alternative embodiment, the muscle specific control element is the MHCK7 enhancer-promoter comprising or consisting of a sequence set forth in SEQ ID NO: 7. In some embodiments, the muscle specific control element comprises a sequence that is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 7.

In embodiments where the packaged vector genome comprises one or more intron sequences, the intron may be selected from, a SV40 Small T intron, a rabbit hemoglobin subunit beta (rHBB) intron, a human β-globin/IgG chimeric intron, a human beta globin IVS2 intron, and an hFIX intron.

In embodiments where the packaged vector genome comprises a polyadenylation signal sequence, the polyadenylation signal sequence may be selected from a synthetic polyadenylation signal sequence, an SV40 polyadenylation signal sequence, a bovine growth hormone (BGH) polyadenylation signal sequence, and a rabbit beta globin polyadenylation signal sequence. In an exemplary embodiment, the polyadenylation signal sequence is a synthetic polyadenylation signal comprising or consisting of a sequence set forth in SEQ ID NO: 9. In some embodiments, the polyadenylation signal sequence is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 9.

In some embodiments, the AAV capsid is from an AAV of serotype 1, 2, 3, 4, 5, 6, 7, 8, 9, 10, 11, 12, rh10, rh74, hu37 (i.e., AAV1, AAV2, AAV3, AAV4, AAV5, AAV6, AAV7, AAV9, AAV10, AAV11, AAV12, AAVrh10, AAVrh74, AAVhu37), or an engineered variant thereof. In an exemplary embodiment, the AAV capsid is selected from an AAV serotype hu37 (AAVhu37) capsid, an AAV serotype 9 (AAV9) capsid, an AAV9 variant capsid, an AAV serotype 8 (AAV8) capsid, or an AAV8 variant capsid. In a further exemplary embodiment, the AAV capsid is the AAVhu37 capsid.

In yet another aspect, the present disclosure provides recombinant adeno-associated virus (rAAV) useful as agents for gene therapy in the treatment of a muscular dystrophy, e.g., DMD, wherein said rAAV comprises an AAV capsid and a vector genome packaged therein. In some embodiments, the AAV capsid is from an AAV of serotype 1, 2, 3, 4, 5, 6, 7, 8, 9, 10, 11, 12, rh10, rh74, hu37 (i.e., AAV1, AAV2, AAV3, AAV4, AAV5, AAV6, AAV7, AAV9, AAV10, AAV11, AAV12, AAVrh10, AAVrh74. AAVhu37), or an engineered variant thereof. In an exemplary embodiment, the AAV capsid is selected from an AAV serotype hu37 (AAVhu37) capsid, an AAVhu37 variant capsid, an AAV serotype 9 (AAV9) capsid, an AAV9 variant capsid, an AAV serotype 8 (AAV8) capsid, or an AAV8 variant capsid. In a further exemplary embodiment, the AAV capsid is the AAVhu37 capsid or an engineered variant thereof. In some embodiments, the packaged vector genome comprises a nucleic acid encoding a micro-dystrophin protein, wherein the nucleic acid comprises a sequence that is at least 99% (e.g., from 99% to 100%) identical to SEQ ID NO: 1. In another embodiment, the packaged vector genome comprises a polynucleotide sequence which is at least 99% (e.g., from 99% to 100%) identical to SEQ ID NO: 3.

In yet another aspect, the present disclosure provides the use of an rAAV disclosed herein for the treatment of a muscular dystrophy, e.g. DMD, wherein said rAAV comprises an AAV capsid and a vector genome packaged therein. In some embodiments, the AAV capsid is from an AAV of serotype 1, 2, 3, 4, 5, 6, 7, 8, 9, 10, 11, 12, rh10, rh74, hu37 (i.e., AAV1, AAV2, AAV3, AAV4. AAV5. AAV6, AAV7, AAV9, AAV10, AAV11, AAV12, AAVrh10, AA Vrh74, AAVhu37), or an engineered variant thereof. In an exemplary embodiment, the AAV capsid is selected from an AAV serotype hu37 (AAVhu37) capsid, an AAVhu37 variant capsid, an AAV serotype 9 (AAV9) capsid, an AAV9 variant capsid, an AAV serotype 8 (AAV8) capsid, or an AAV8 variant capsid. In a further exemplary embodiment, the AAV capsid is the AAVhu37 capsid or an engineered variant thereof. In some embodiments, packaged vector genome comprises a nucleic acid encoding a micro-dystrophin protein, wherein the nucleic acid comprises a sequence that is at least 99% (e.g., from 99% to 100%) identical to SEQ ID NO: 1. In another embodiment, the packaged vector genome comprises a polynucleotide sequence which is at least 99% (e.g., from 99% to 100%) identical to SEQ ID NO: 3.

The present disclosure further relates to pharmaceutical compositions comprising a synthetic nucleic acid sequence or an rAAV disclosed herein. In some embodiments, the pharmaceutical composition comprises a synthetic nucleic acid and a pharmaceutically acceptable carrier or excipient. In some embodiments, the pharmaceutical composition comprises an rAAV and a pharmaceutically acceptable carrier or excipient. In some embodiments, the pharmaceutical composition comprising an rAAV is formulated for intravenous, subcutaneous, intramuscular, intradermal, intraperitoneal, or intrathecal administration. In an exemplary embodiment, the pharmaceutical composition comprising an rAAV is formulated for intravenous or intramuscular administration.

In yet another aspect, the present disclosure provides methods of treating muscular dystrophy in a human subject comprising administering to the human subject a therapeutically effective amount of at least one rAAV disclosed herein. In one embodiment, the muscular dystrophy is caused by a mutation in dystrophin. In one embodiment, the muscular dystrophy is selected from Duchenne muscular dystrophy (DMD), Becker muscular dystrophy, and X-linked dilated cardiomyopathy.

In one embodiment, the present disclosure provides a muscular dystrophy, e.g., DMD, comprising administering an rAAV that includes an AAV capsid and a vector genome packaged therein, wherein the nucleic acid comprises a sequence that is at least 99% (e.g., from 99% to 100%) identical to SEQ ID NO: 1. In some embodiments, the method may further comprise administration of an IgG-degrading protease (e.g., the Streptococcus pyogenes IdeS or the Streptococcus equi IdeZ) prior to administration of the rAAV. In some embodiments, the present disclosure provides a method of treating a muscular dystrophy. e.g., DMD, in a human subject comprising administering a therapeutically effective amount of at least one rAAV disclosed herein, wherein the human subject has been administered an IgG-degrading protease.

In certain embodiments, the present disclosure provides methods of treating a muscular dystrophy, e.g., DMD, in human subject comprising administering to a human subject diagnosed with at least one mutation in dystrophin a therapeutically effective amount of at least one rAAV disclosed herein. In one embodiment, the present disclosure provides a muscular dystrophy, e.g., DMD, in human subject comprising administering to a human subject diagnosed with at least one mutation in dystrophin comprising administering an rAAV that includes an AAV capsid and a vector genome packaged therein, wherein the packaged vector genome comprises a nucleic acid encoding a micro-dystrophin protein, wherein the nucleic acid comprises a sequence that is at least 99% (e.g., from 99% to 100%) identical to SEQ ID NO: 1. In some embodiments, the packaged vector genome comprises a polynucleotide sequence which is at least 99% (e.g., from 99% to 100%) identical to SEQ ID NO: 3. In some embodiments, the capsid is an AAVhu37 capsid.

In some embodiments, the rAAV is administered intravenously, subcutaneously, intramuscularly, intradermally, intraperitoneally, or intrathecally. In an exemplary embodiment, the rAAV is administered intravenously or intramuscularly. In some embodiments, the rAAV is administered at a dose of about 1×10¹² genome copies (GC)/kg to about 1×10¹⁶ genome copies (GC)/kg. In further embodiments, the rAAV is administered at a dose of about 1×10¹³ genome copies (GC)/kg to about 1×10¹⁵ genome copies (GC)/kg. In further embodiments, the rAAV is administered at a dose at or about 1×10¹³ GC/kg, at or about 1×10¹⁴ GC/kg, at or about 2×10¹⁴ GC/kg, at or about 3×10¹⁴ GC/kg, at or about 4×10¹⁴ GC/kg, at or about 5×10¹⁴ GC/kg, at or about 6×10¹⁴ GC/kg, at or about 7×10¹⁴ GC/kg, at or about 8×10¹⁴ GC/kg, at or about 9×10¹⁴ GC/kg, or at or about 1×10¹⁵ GC/kg. In some embodiments, a single dose of rAAV is administered. In other embodiments, multiple doses of rAAV are administered.

In some aspects, provided herein are host cells comprising a synthetic nucleic acid molecule, an AAV vector, or an rAAV disclosed herein. In specific embodiments, the host cells may be suitable for the propagation of AAV. In certain embodiments, the host cell is selected from a HeLa, Cos-7, HEK293, A549. BHK. Vero, RD, HT-1080, ARPE-19, and MRC-5 cell. In some embodiments, the host cell is a HeLa cell which has been engineered to inactivate one or more endogenous genes.

These and other aspects and features of the invention are described in the following sections of the present disclosure.

### BRIEF DESCRIPTION OF THE DRAWINGS

The invention can be more completely understood with reference to the following drawings.

FIG. 1 is an illustrative diagram showing an exemplary packaged vector genome construct comprising a coding sequence for a micro-dystrophin protein under the control of a CK8 promoter. The packaged vector genome comprises from 5″ to 3′: a 5′-inverted terminal repeat (5′-ITR), a CK8 promoter, an intron, a coding sequence for a human micro-dystrophin, a synthetic polyadenylation signal sequence, and a 3′-inverted terminal repeat (3′-ITR). This diagram illustrates the configuration of SEQ ID NO: 3 (4,784 bp), which contains the synthetic nucleic acid set forth in SEQ ID NO: 1 (3,810 bp) that codes for a human micro-dystrophin protein.

FIG. 2 is a bar graph showing a comparison of MD5 micro-dystrophin protein expression from a native MD5 coding sequence (SEQ ID NO: 8) and a synthetic codon-optimized coding sequence (SEQ ID NO: 1) in an in vitro system. The synthetic codon-optimized coding sequence demonstrated a statistically significant (p<0.0001), approximately 7-fold increase in protein expression compared to the native coding sequence (n=2). Micro-dystrophin protein expression levels were determined via Meso Scale Discovery (MSD) ELISA utilizing a commercially available antibody specific for human dystrophin. Raw MSD ELISA reads were normalized to the native micro-dystrophin group mean value and expressed as fold change.

### DETAILED DESCRIPTION OF THE INVENTION

This invention provides a range of novel agents and compositions to be used for therapeutic applications. The nucleic acid sequences, vectors, recombinant viruses, and associated compositions of this invention can be used for ameliorating, preventing, or treating various muscular dystrophies as described herein.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.). The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers. Inc., 1995 (ISBN 1-56081-569-8).

In order to facilitate review of the various embodiments of the disclosure, the following explanations of specific terms are provided:

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 12 recognized serotypes of AAV (AAV1-12) and several AAV serotype variants such as AAVhu37, AAVrh10, and AAVrh74.

Administration/Administer: To provide or give a subject an agent, such as a therapeutic agent (e.g., a recombinant AAV), by any effective route. Exemplary routes of administration include, but are not limited to, injection (such as intravenous, subcutaneous, intramuscular, intradermal, intraperitoneal, or intrathecal administration), oral, intraductal, sublingual, rectal, transdermal, intranasal, vaginal and inhalation routes.

Coding Sequence: A “coding sequence” means the nucleotide sequence encoding a polypeptide in vitro or in vivo when operably linked to appropriate regulatory sequences. The coding sequence may or may not include regions preceding and following the coding region. e.g., 5′ untranslated (5′ UTR) and 3′ untranslated (3′ UTR) sequences, as well as intervening sequences (introns) between individual coding segments (exons).

Codon-optimized: A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

Enhancer: A nucleic acid sequence that increases the rate of transcription by increasing the activity of a promoter.

Intron: A stretch of DNA within a gene that does not contain coding information for a protein. Introns are removed before translation of a messenger RNA.

Inverted terminal repeat (ITR): Symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV integrating vectors.

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carrier: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease (such as DMD) refers to inhibiting the full development of a disease. “Treating” refers to a therapeutic intervention that ameliorates a sign or symptom of a disease or pathological condition (such as DMD) after it has begun to develop. “Ameliorating” refers to the reduction in the number or severity of signs or symptoms of a disease (such as DMD).

Promoter: A region of DNA that directs/initiates transcription of a nucleic acid (e.g., a gene). A promoter includes necessary nucleic acid sequences near the start site of transcription. Many promoter sequences are known to the person skilled in the art and even a combination of different promoter sequences in artificial nucleic acid molecules is possible.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

Similarly, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein or virus. As used herein, “recombinant AAV” refers to an AAV particle in which a recombinant nucleic acid molecule such as a recombinant nucleic acid molecule encoding micro-dystrophin has been packaged.

Sequence identity: The identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. A commonly used tool is the NCBI Basic Local Alignment Search Tool (BLAST) (Altschul et al., J. Mol. Biol. 215:403-10, 1990), which is available from several sources, including the National Center for Biological Information (NCBI) and on the internet, for use in connection with the sequence analysis programs blastp, blastn, blastx, tblastn and tblastx. Additional information can be found at the NCBI web site.

Serotype: A group of closely related microorganisms (such as viruses) distinguished by a characteristic set of antigens.

Subject: Living multi-cellular vertebrate organisms, a category that includes human and non-human mammals. In some embodiments, the subject is a human. In an exemplary embodiment, the human subject is a pediatric subject. i.e., a human subject of ages 0-18 years old inclusive. Alternatively, the human subject is an adult subject, i.e., a human subject greater than 18 years old. In some embodiments, the subject (e.g., human subject) has been administered a corticosteroid prior to treatment with an rAAV of the invention. In some embodiments, the subject (e.g., human subject) has been administered an IgG-degrading protease prior to treatment with an rAAV of the invention. In some embodiments, the subject (e.g., human subject) has been administered a corticosteroid and has also been administered an IgG-degrading protease prior to treatment with an rAAV of the invention.

Synthetic: A synthetic nucleic acid is a non-naturally occurring nucleic acid sequence which can be chemically synthesized in a laboratory and/or expressed by a recombinant microorganism or recombinant virus, e.g., a recombinant AAV.

Therapeutically effective amount: A quantity of a specified pharmaceutical or therapeutic agent (e.g., a recombinant AAV) sufficient to achieve a desired effect in a subject, or in a cell, being treated with the agent. The effective amount of the agent will be dependent on several factors, including, but not limited to the subject or cells being treated, and the manner of administration of the therapeutic composition.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an AAV vector.

Unless otherwise explained, all technical and scientific terms used herein have the same meaning as commonly understood by one of ordinary skill in the art to which this disclosure belongs. The singular terms “a.” “an.” and “the” include plural referents unless context clearly indicates otherwise “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Novel Synthetic Nucleic Acids Encoding Micro-Dystrophin:

This invention provides compositions and methods of their use in gene therapy. Amongst the compositions described herein are synthetic nucleic acids encoding a micro-dystrophin protein. Accordingly, in a first aspect, the present disclosure provides novel synthetic nucleic acid sequences encoding a micro-dystrophin protein.

In some embodiments, the micro-dystrophin protein encoded by a novel synthetic nucleic acid described herein is a micro-dystrophin protein termed “MD5” (SEQ ID NO: 10; 1.270 amino acids, which is listed as SEQ ID NO: 4 in U.S. Pat. No. 10,479,821) or a functional fragment or functional variant thereof. As described in U.S. Pat. No. 10,479,821 (incorporated herein by reference in its entirety), MD5-which is also termed “μDys5” or “micro-Dys5”-comprises an amino-terminal actin-binding domain; a β-dystroglycan binding domain; and a spectrin-like repeat domain, consisting of five spectrin-like repeats, including spectrin-like repeat 1 (SR1), spectrin-like repeat 16 (SR16), spectrin-like repeat 17 (SR17), spectrin-like repeat 23 (SR23), and spectrin-like repeat 24 (SR24).

In some embodiments, the present disclosure provides synthetic nucleic acids encoding the MD5 micro-dystrophin protein or a functional fragment or a functional variant thereof. In one embodiment, the synthetic nucleic acid encoding the MD5 micro-dystrophin protein or a functional fragment or a functional variant thereof comprises a sequence that is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 1. In an exemplary embodiment, the synthetic nucleic acid encoding the MD5 micro-dystrophin protein or a functional fragment or functional variant thereof comprises a sequence that is at least 99% (e.g., from 99% to 100%) identical to SEQ ID NO: 1. In a further exemplary embodiment, the synthetic nucleic acid encodes the MD5 micro-dystrophin protein and comprises or consists of the sequence set forth in SEQ ID NO: 1. In some embodiments, the synthetic nucleic acid encoding a MD5 micro-dystrophin protein, e.g., SEQ ID NO: 1 may further comprise a stop codon (TGA, TAA, or TAG) at the 3′ end, e.g., such as the sequence exemplified set forth in SEQ ID NO: 2 that includes a TAG stop codon at the 3′ end of SEQ ID NO: 1.

In some embodiments, the present disclosure also provides fragments of the nucleic acid sequence shown in SEQ ID NO: 1 which encode a fragment of the MD5 polypeptide having functional micro-dystrophin activity. In some embodiments, the present disclosure provides fragments of the nucleic acid sequence shown in SEQ ID NO: 1 which encode at least 300, at least 400, at least 500, at least 600, at least 700, at least 800, at least 900, at least 1000, at least 1100, at least 1200, at least 1250, at least 1260, or at least 1265 contiguous amino acid residues of SEQ ID NO: 10, and wherein the fragment of the MD5 polypeptide retains one or more activities associated with the full-length MD5 micro-dystrophin polypeptide of SEQ ID NO: 10 (e.g., nNOS binding activity) Such fragments may be obtained by recombinant techniques that are routine and well-known in the art. In some embodiments, the present disclosure provides fragments of the nucleic acid sequence shown in SEQ ID NO: 1 which are at least 900, at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, at least 2500, at least 2600, at least 2700, at least 2800, at least 2900, at least 3000, at least 3100, at least 3200, at least 3300, at least 3400, at least 3500, at least 3600, at least 3700, or at least 3800 contiguous nucleotides of SEQ ID NO: 1.

In some embodiments, the present disclosure also provides synthetic nucleic acid sequences which encode variants of the MD5 micro-dystrophin polypeptide of SEQ ID NO: 10. In some embodiments, the variant polypeptides may be at least 80% (e.g., from 80% to 100%, such as 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or 100%) identical to SEQ ID NO: 10. In some embodiments, the variant polypeptides are therapeutic in nature. In some embodiments, the variant therapeutic polypeptides may have at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least at least 24, at least 25, at least 26, at least 27, at least 28, at least 29, at least 30, at least 31, at least 32, at least 33, at least 34, at least 35, at least 36, at least 37, at least 38, at least 39, or at least 40 different residues as compared to SEQ ID NO: 10. Such variants may be obtained by recombinant techniques that are routine and well-known in the art. In some embodiments, the present disclosure provides synthetic nucleic acid variants of SEQ ID NO: 1 which encode variants of the MD5 micro-dystrophin polypeptide of SEQ ID NO: 10. Such variants of SEQ ID NO: 1 may comprise one or more nucleotide changes relative to SEQ ID NO: 1 and encode a variant of the MD5 micro-dystrophin polypeptide of SEQ ID NO: 10.

In various aspects described herein, the invention contemplates the use of rAAV to deliver fragments, variants, or fusions of the MD5 micro-dystrophin polypeptide. In exemplary embodiments, the fragment, variant, or MD5 portion of a fusion protein is encoded by a corresponding fragment, variant, or MD5-encoding portion of a sequence which is at least 99% (e.g., from 99% to 100%) identical to SEQ ID NO: 1.

### Novel Vector Genome Constructs Encoding Micro-Dystrophin:

In another aspect, the present disclosure provides novel vector genome constructs useful in the treatment of muscular dystrophy, e.g., DMD, Becker muscular dystrophy, or X-linked cardiomyopathy.

In some embodiments, the vector genome construct comprises a nucleic acid sequence encoding a micro-dystrophin protein. In some embodiments, the vector genome construct further comprises one or more additional sequence elements selected from: (a) a 5′-inverted terminal repeat (ITR); (b) a muscle specific control element; (c) an intron; (d) a polyadenylation signal sequence; and (e) a 3-inverted terminal repeat (ITR). In an exemplary embodiment, the vector genome construct comprises: (i) a nucleic acid sequence encoding the MD5 micro-dystrophin protein (SEQ ID NO: 10), or a fragment, variant, or fusion protein thereof; and (ii) one or more additional sequence elements selected from: (a) a 5′-inverted terminal repeat (ITR); (b) a muscle specific control element; (c) an intron; (d) a polyadenylation signal sequence; and (e) a 3′-inverted terminal repeat (ITR).

In some embodiments, the vector genome construct comprises: (i) a micro-dystrophin coding sequence that is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 1; and (ii) one or more additional sequence elements selected from: (a) a 5′-inverted terminal repeat (ITR); (b) a muscle specific control element; (c) an intron; (d) a polyadenylation signal sequence; and (e) a 3″-inverted terminal repeat (ITR). In an exemplary embodiment, the vector genome construct comprises: (i) a micro-dystrophin coding sequence that is at least 99% (e.g., from 99% to 100%) identical to SEQ ID NO: 1; and (ii) one or more additional sequence elements selected from: (a) a 5′-inverted terminal repeat (ITR); (b) a muscle specific control element; (c) an intron; (d) a polyadenylation signal sequence; and (e) a 3″-inverted terminal repeat (ITR). In a further exemplary embodiment, the vector genome construct comprises: (i) a micro-dystrophin coding sequence set forth in SEQ ID NO: 1; and (ii) one or more additional sequence elements selected from: (a) a 5′-inverted terminal repeat (ITR); (b) a muscle specific control element; (c) an intron; (d) a polyadenylation signal sequence; and (c) a 3′-inverted terminal repeat (ITR).

In some embodiments, the vector genome construct comprises a sequence which is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 3. In an exemplary embodiment, the vector genome construct comprises a sequence which is at least 99% (e.g., from 99% to 100%) identical to SEQ ID NO: 3. In a further exemplary embodiment, the vector genome construct comprises or consists of the sequence set forth in SEQ ID NO: 3.

Recombinant AAV (rAAV):

In yet another aspect, the present disclosure provides novel recombinant adeno-associated virus (rAAV) comprising an adeno-associated virus (AAV) capsid and a vector genome packaged therein, wherein said packaged vector genome comprises a nucleic acid encoding a micro-dystrophin protein.

In some embodiments, the rAAV comprises an AAV capsid and a packaged vector genome, wherein the packaged vector genome comprises: (i) a micro-dystrophin coding sequence that is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 1; and optionally (ii) one or more additional sequence elements selected from: (a) a 5′-inverted terminal repeat (ITR); (b) a muscle specific control element; (c) an intron; (d) a polyadenylation signal sequence; and (e) a 3-inverted terminal repeat (ITR). In an exemplary embodiment, the rAAV comprises an AAV capsid and a packaged vector genome, wherein the packaged vector genome comprises: (i) a micro-dystrophin coding sequence that is at least 99% (e.g., from 99% to 100%) identical to SEQ ID NO: 1; and optionally (ii) one or more additional sequence elements selected from: (a) a 5′-inverted terminal repeat (ITR); (b) a muscle specific control element; (c) an intron; (d) a polyadenylation signal sequence; and (e) a 3′-inverted terminal repeat (ITR). In a further exemplary embodiment, the rAAV comprises an AAV capsid and a packaged vector genome, wherein the packaged vector genome comprises: (i) a micro-dystrophin coding sequence set forth in SEQ ID NO: 1; and optionally (ii) one or more additional sequence elements selected from: (a) a 5′-inverted terminal repeat (ITR); (b) a muscle specific control element; (c) an intron; (d) a polyadenylation signal sequence; and (e) a 3′-inverted terminal repeat (ITR).

In some embodiments, the rAAV comprises an AAV capsid and a packaged vector genome, wherein the packaged vector genome comprises a sequence which is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 3. In an exemplary embodiment, the rAAV comprises an AAV capsid and a packaged vector genome, wherein the packaged vector genome comprises a sequence which is at least 99% (e.g., from 99% to 100%) identical to SEQ ID NO: 3. In a further exemplary embodiment, the rAAV comprises an AAV capsid and a packaged vector genome, wherein the packaged vector genome comprises or consists of a sequence set forth in SEQ ID NO: 3.

### Inverted Terminal Repeats (ITRs):

In some embodiments, the rAAV comprises a packaged vector genome which comprises an AAV ITR sequence, which functions as both the origin of vector DNA replication and the packaging signal of the vector genome, when AAV and adenovirus helper functions are provided in trans. Additionally, the ITRs serve as the target for single-stranded endonucleatic nicking by the large Rep proteins, resolving individual genomes from replication intermediates.

In some embodiments, the 5′-ITR is derived from AAV2. In some embodiments, the 5′-ITR comprises or consists of SEQ ID NO: 4 (in the plus/plus strand orientation). In other embodiments, the 5′-ITR is from a non-AAV2 source.

In some embodiments, the 3′-ITR is derived from AAV2. In some embodiments, the 3′-ITR comprises or consists of SEQ ID NO: 4 (in the plus/minus strand orientation), which corresponds to SEQ ID NO: 5 in the plus/plus strand orientation. In other embodiments, the 3′-ITR is from a non-AAV2 source.

In some embodiments, both the 5′-ITR and the 3′-ITR are derived from AAV2. In other embodiments, the 5′-ITR and the 3″-ITR are both from a non-AAV2 source.

### Muscle Specific Control Element:

In some embodiments, the rAAV comprises a packaged vector genome which comprises a muscle specific control element that helps drive and/or regulate micro-dystrophin expression. In an exemplary embodiment, the muscle specific control element is located between a 5′-ITR sequence and the coding sequence for a micro-dystrophin protein. In some embodiments, the muscle specific control element is located upstream of an intron sequence. In some embodiments, the muscle specific control element is a promoter. In some embodiments, the muscle specific control element is an enhancer. In some embodiments, the muscle specific control element is a promoter/enhancer.

In some embodiments, the muscle specific control element is selected from a CK8 promoter, a CK7 promoter, a CK9 promoter, a muscle specific creatine kinase (MCK) promoter, truncated MCK (tMCK), myosin heavy chain (MHC), a hybrid α-myosin heavy chain enhancer-/MCK (MHCK7) enhancer-promoter, a human skeletal actin gene element, a cardiac actin gene element, a myocyte-specific enhancer binding factor mef, C5-12, a murine creatine kinase enhancer element, a skeletal fast-twitch troponin e gene element, a slow-twitch cardiac troponin c gene element, the slow-twitch troponin i gene element, a hypoxia-inducible nuclear factor (e.g., HIF-1α, HIF-1β, HIF-2a, HIF-2β, HIF-3α, and HIF-3β), a steroid-inducible element, and a glucocorticoid response element (gre).

In an exemplary embodiment, the muscle specific control element is a CK8 promoter comprising or consisting of a sequence set forth in SEQ ID NO: 6. In some embodiments, the muscle specific control element comprises a sequence that is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 6.

In another embodiment, the muscle specific control element is a hybrid α-myosin heavy chain enhancer-/MCK (MHCK7) enhancer-promoter comprising or consisting of a sequence set forth in SEQ ID NO: 7. In some embodiments, the muscle specific control element comprises a sequence that is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 7.

### Intron:

In some embodiments, the rAAV contains a packaged vector genome that comprises one or more intron sequences. Various introns are known in the art. Introns may facilitate processing of the RNA transcript in mammalian host cells, increase expression of the protein of interest (e.g., a micro-dystrophin), and/or optimize packaging of the vector into AAV particles. In some embodiments, the rAAV contains a packaged vector genome that comprises a synthetic intron sequence. In some embodiments, the rAAV contains a packaged vector genome that comprises a naturally-occurring intron sequence.

In some embodiments, the intron is located between a promoter and/or enhancer sequence and the coding sequence for a micro-dystrophin protein. In some embodiments, the intron is located upstream of the coding sequence for a micro-dystrophin protein.

In some embodiments, the intron is selected from a SV40 Small T intron, a rabbit hemoglobin subunit beta (rHBB) intron, a human β-globin/IgG chimeric intron, a human beta globin IVS2 intron, and an hFIX intron. In other embodiments, an intron sequence from a gene encoding a protein selected from eukaryotic translation initiation factor 2, subunit 1 (EIF2S1), collagen type I alpha 2 chain (COL1A2), secreted protein acidic and rich in cysteine (SPARC), signal transducer and activator of transcription 3 (STAT3), enolase 1 (ENO1), pyruvate kinase (PKM), aldolase fructose-bisphosphate A (ALDOA), Y-box binding protein 1 (YBX1), guanine nucleotide binding protein {G protein}, beta polypeptide 2-like 1 (GNB2L1), ribosomal protein S3 (RPS3), GNAS complex locus (GNAS), filamin A (FLNA), transferrin receptor (TFRC), polyA binding protein cytoplasmic 1 (PABPC1), ubiquitin like modifier activating enzyme 1 (UBA1), calnexin (CANX), and lactate dehydrogenase A (LDHA) may be used.

### Polyadenylation Signal:

In some embodiments, the rAAV contains a packaged vector genome that comprises a polyadenylation signal sequence. Various polyadenylation signal sequences are known in the art. Polyadenylation signal sequences help provide effective termination of transcription and stabilize transcribed mRNA. In some embodiments, the rAAV contains a packaged vector genome that comprises a synthetic polyadenylation signal sequence. In some embodiments, the rAAV contains a packaged vector genome that comprises a naturally-occurring polyadenylation signal sequence.

In some embodiments, the polyadenylation signal sequence is located is between the coding sequence for a micro-dystrophin protein and a 3′-ITR. In some embodiments, the polyadenylation signal sequence is located immediately downstream of the coding sequence for a micro-dystrophin protein.

In some embodiments, the polyadenylation signal sequence may be selected from a synthetic polyadenylation signal sequence, an SV40 polyadenylation signal sequence, a bovine growth hormone (BGH) polyadenylation signal sequence, and a rabbit beta globin polyadenylation signal sequence.

In an exemplary embodiment, the rAAV contains a packaged vector genome that comprises a synthetic polyadenylation signal sequence. In one embodiment, the synthetic polyadenylation signal sequence comprises or consists of the sequence set forth in SEQ ID NO: 9.

In some embodiments, the rAAV contains a packaged vector genome that comprises a polyadenylation signal sequence comprising a sequence is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 9.

rAAV Capsid:

In various embodiments described herein, the rAAV comprises an AAV capsid. The AAV capsid can be from an AAV of serotype 1, 2, 3, 4, 5, 6, 7, 8, 9, 10, 11, 12, rh10, rh74, hu37 (i.e., AAV1, AAV2, AAV3, AAV4, AAV5, AAV6, AAV7, AAV8, AAV9, AAV10, AAV11, AAV12. AAVrh10, AAVrh74, AAVhu37), as well as any one of the more than 100 naturally occurring variants isolated from human and nonhuman primate tissues. See, e.g., Choi et al., 2005. Carr Gene Ther. 5: 299-310, 2005 and Gao et al., 2005, Curr Gene Ther. 5:285-297.

In certain exemplary embodiments, the rAAV administered according to the invention comprises an AAVhu37 capsid. The AAVhu37 capsid is a self-assembled AAV capsid composed of multiple AAVhu37 vp proteins. The AAVhu37 vp proteins (vp1, vp2, and vp3) are typically expressed as alternative mRNA splice variants. The AAVhu37 vp1 amino acid sequence is set forth in SEQ ID NO: 12 (GenBank Accession: AAS99285.1). In some embodiments, the AAVhu37 vp1 amino acid sequence of SEQ ID NO: 12 is encoded by SEQ ID NO: 11, or a sequence at least 95% (e.g., from 95% to 100%, such as 95%, 96%, 97%, 98%, 99%, or 100%) identical thereto.

In some embodiments, the rAAV administered according to the invention may comprise an AAV capsid comprising a vp1 amino acid sequence that is at least 95%, at least 96%, at least 97%, at least 98%, at least 99%, at least 99.5%, at least 99.9%, or more identical to SEQ ID NO: 12. In some embodiments, the rAAV administered according to the invention comprises a capsid comprising the vp1 amino acid sequence of SEQ ID NO: 12.

In some embodiments, the rAAV administered according to the invention may comprise an AAV capsid comprising a vp1 amino acid sequence that is at least 95%, at least 96%, at least 97%, at least 98%, at least 99%, at least 99.5%, at least 99.9%, or more identical to a vp1 amino acid sequence of serotype 1, 2, 3, 4, 5, 6, 7, 8, 9, 10, 11, 12, rh10, or rh74.

Also included within the scope of the invention are variant AAV capsids which have been engineered to harbor one or more beneficial therapeutic properties (e.g., improved targeting for select tissues, increased ability to evade the immune response, reduced stimulation of neutralizing antibodies, etc.). Non-limiting examples of such engineered variant capsids are described in U.S. Pat. Nos. 9,506,083, 9,585,971, 9,587,282, 9,611,302, 9,725,485, 9,856,539, 9,909,142, 9,920,097, 10,011,640, 10,081,659, 10,179,176, 10,202,657, 10,214,566, 10,214,785, 10,266,845, 10,294,281, 10,301,648, 10,385,320, and 10,392,632 and in PCT Publication Nos. WO/2017/165859, WO/2018/022905, WO/2018/156654, WO/2018/222503, and WO/2018/226602, the disclosures of which are herein incorporated by reference.

In some embodiments, an AAV variant capsid may have at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least at least 24, at least 25, at least 26, at least 27, at least 28, at least 29, at least 30, at least 31, at least 32, at least 33, at least 34, at least 35, at least 36, at least 37, at least 38, at least 39, or at least 40 different residues as compared to a naturally occurring AAV capsid of serotype 1, 2, 3, 4, 5, 6, 7, 8, 9, 10, 11, 12, rh10, rh74, or hu37.

Beyond the aforementioned capsids. AAV yet to be discovered, or a recombinant AAV based thereon, may be used as a source for the AAV capsid.

In addition, in some embodiments, the AAV capsid may be chimeric, comprising domains from two, or three, or four, or more of the aforementioned AAV capsid proteins. In some embodiments, the AAV capsid is a mosaic of Vp1, Vp2, and Vp3 monomers from two or three different AAVs or recombinant AAVs. In some embodiments, an rAAV composition comprises more than one of the aforementioned capsids.

### Host Cells Comprising a Recombinant Nucleic Acid Molecule:

In yet another aspect, provided herein are host cells comprising a recombinant nucleic acid molecule, viral vector, e.g., an AAV vector, or an rAAV disclosed herein. In specific embodiments, the host cells may be suitable for the propagation of AAV.

In one embodiment, provided herein is a host cell comprising a recombinant nucleic acid molecule set forth in SEQ ID NO: 1. In another embodiment, provided herein is a host cell comprising a recombinant nucleic acid molecule that is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 1.

In one embodiment, provided herein is a host cell comprising a recombinant nucleic acid molecule set forth in SEQ ID NO: 3. In another embodiment, provided herein is a host cell comprising a recombinant nucleic acid molecule that is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 3.

A vast range of host cells can be used, such as bacteria, yeast, insect, mammalian cells, etc. In some embodiments, the host cell can be a cell (or a cell line) appropriate for production of recombinant AAV (rAAV), for example, a HeLa, Cos-7, HEK293, A549, BHK, Vero, RD, HT-1080, ARPE-19, or MRC-5 cell. In an exemplary embodiment, the host cell is a HeLa cell. In some embodiments, the host cell is a Hela cell which has been engineered to inactivate one or more endogenous genes. In some embodiment, the host cell is a HeLa cell which has been engineered to inactivate one or more endogenous genes described in PCT Publication No. WO/2020/210507, e.g. ATP5EP2, LINC00319, CYP3A7, ABCA10, NOG, RGMA, SPANXN3, PGAS, MYRIP, KCNN2, and NALCN-AS1. In an exemplary embodiment, the endogenous genes are selected from KCNN2 (Potassium Calcium-Activated Channel Subfamily N Member 2) and RGMA (Repulsive Guidance Molecule BMP Co-Receptor A).

The recombinant nucleic acid molecules or vectors can be delivered into the host cell culture using any suitable method known in the art. In some embodiments, a stable host cell line that has the recombinant nucleic acid molecule or vector inserted into its genome is generated. In some embodiments, a stable host cell line is generated, which contains an rAAV vector described herein. After transfection of the rAAV vector to the host culture, integration of the rAAV into the host genome can be assayed by various methods, such as antibiotic selection, fluorescence-activated cell sorting, southern blot, PCR based detection, and fluorescence in situ hybridization.

### Recombinant AAV for Gene Therapy:

In yet another aspect, the present disclosure provides recombinant adeno-associated virus (rAAV) useful as agents for gene therapy in the treatment of a muscular dystrophy, wherein said rAAV comprises an AAV capsid and a vector genome packaged therein. In one embodiment, the muscular dystrophy is caused by a mutation in dystrophin. In some embodiments, the muscular dystrophy is selected from Duchenne muscular dystrophy (DMD), Becker muscular dystrophy, and X-linked dilated cardiomyopathy.

In some embodiments, the rAAV contains a packaged vector genome comprising as operably linked components in 5′ to 3′ order: (a) a 5′-inverted terminal repeat (ITR); (b) a muscle specific control element; (c) an intron; (d) a partial or complete coding sequence for the MD5 micro-dystrophin protein; (e) a polyadenylation signal sequence; and (f) a 3-inverted terminal repeat (ITR).

In some embodiments, the coding sequence for the MD5 micro-dystrophin protein comprises SEQ ID NO: 1, or a sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical thereto.

In some embodiments, the packaged vector genome comprises SEQ ID NO: 3, or a sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical thereto.

In some embodiments, the AAV capsid is selected from an AAV of serotype 8, serotype 9, rh74, and hu37. In an exemplary embodiment, the AAV capsid is an hu37 (AAVhu37) capsid.

In some exemplary embodiments, the rAAV comprises an AAVhu37 capsid and a vector genome packaged therein, wherein the packaged vector genome comprises as operably linked components in 5′ to 3′ order: (a) an AAV2 5′-ITR; (b) CK8 promoter; (c) an intron; (d) a coding sequence for a human micro-dystrophin comprising the sequence set forth in SEQ ID NO: 1; (e) a synthetic polyadenylation signal sequence comprising the sequence set forth in SEQ ID NO: 9; and (f) an AAV2 3′-ITR.

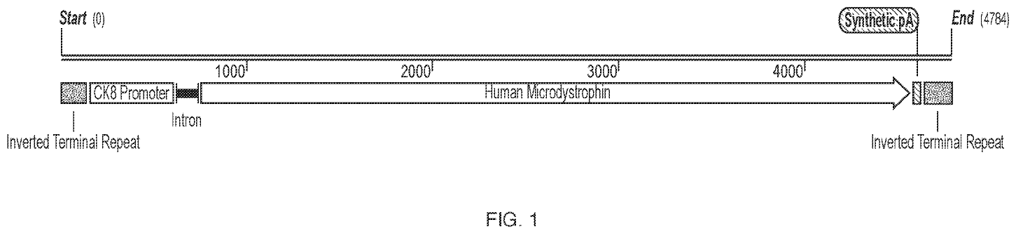

An illustrative diagram showing an exemplary packaged vector genome construct for the expression of the MD5 micro-dystrophin protein is provided in FIG. 1, which shows in 5′ to 3′ order: a 5′-ITR, a CK8 promoter, an intron, a coding sequence for the MD5 micro-dystrophin protein, a synthetic polyadenylation signal sequence, and a 3′-ITR. Illustrated in FIG. 1 is the configuration of SEQ ID NO: 3 (4,784 bp), which contains the synthetic nucleic acid set forth in SEQ ID NO: 1 (3,810 bp) that codes for a human micro-dystrophin protein (MD5).

### Pharmaceutical Compositions:

In yet another aspect, the present disclosure provides a pharmaceutical composition comprising a synthetic nucleic acid sequence or an rAAV disclosed herein. In some embodiments, the pharmaceutical composition comprises a synthetic nucleic acid and a pharmaceutically acceptable carrier or excipient. In some embodiments, the pharmaceutical composition comprises an rAAV and a pharmaceutically acceptable carrier or excipient.

In certain exemplary embodiments, the present disclosure provides a pharmaceutical composition that comprises an rAAV of the invention (e.g., an rAAV for the delivery of micro-dystrophin) and a pharmaceutically acceptable carrier or excipient. In some embodiments, the pharmaceutical composition comprising an rAAV of the invention (e.g., an rAAV for the delivery of micro-dystrophin) is formulated for intravenous, subcutaneous, intramuscular, intradermal, intraperitoneal, or intrathecal administration. In an exemplary embodiment, the pharmaceutical composition comprising an rAAV is formulated for intravenous or intramuscular administration.

In some embodiments, the pharmaceutical composition comprising an rAAV of the invention (e.g., an rAAV for the delivery of micro-dystrophin) may be formulated for local administration or systemic administration. For example, systemic administration is administration into the circulatory system so that the entire body is affected. Systemic administration includes enteral administration such as absorption through the gastrointestinal tract and parental administration through injection, infusion or implantation.

In some embodiments, the rAAV is formulated in a buffer/carrier suitable for infusion in human subjects. The buffer/carrier should include a component that prevents the rAAV from sticking to the infusion tubing but does not interfere with the rAAV binding activity in vivo. Various suitable solutions may include one or more of a buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration. The pH may be in the range of 6.5 to 8.5, or 7 to 8.5, or 7.5 to 8. A suitable surfactant, or combination of surfactants, may be selected from among Poloxamers. i.e., nonionic triblock copolymers composed of a central hydrophobic chain of polyoxypropylene 10 (poly(propylene oxide)) flanked by two hydrophilic chains of polyoxyethylene (poly(ethylene oxide)). SOLUTOL HS 15 (Macrogol-15 Hydroxystearate). LABRASOL (Polyoxy capryllic glyceride), polyoxy 10 oleyl ether. TWEEN (polyoxyethylene sorbitan fatty acid esters), ethanol and polyethylene glycol.

In an exemplary embodiment, the rAAV is formulated in a solution comprising NaCl (e.g., 200 mM NaCl), MgCl₂ (e.g., 1 mM MgCl₂), Tris (e.g., 20 mM Tris), pH 8.0, and poloxamer 188 (e.g., 0.005% or 0.01% poloxamer 188).

### Methods of Treating Muscular Dystrophy:

In yet another aspect, the present disclosure provides methods of treating a muscular dystrophy in a human subject comprising administering to the human subject a therapeutically effective amount of at least one synthetic nucleic acid sequence disclosed herein.

In yet another aspect, the present disclosure provides methods of treating a muscular dystrophy in a human subject comprising administering to the human subject a therapeutically effective amount of at least rAAV disclosed herein.

In some embodiments, the muscular dystrophy is caused by a mutation in dystrophin. In some embodiments, the muscular dystrophy is selected from Duchenne muscular dystrophy (DMD), Becker muscular dystrophy, and X-linked dilated cardiomyopathy.

In one embodiment, the present disclosure provides a method of treating a muscular dystrophy (e.g., DMD) comprising administering an rAAV that comprises an AAV capsid and a vector genome packaged therein, wherein the packaged vector genome comprises: (i) a micro-dystrophin coding sequence that is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 1; and optionally (ii) one or more additional sequence elements selected from: (a) a 5′-inverted terminal repeat (ITR); (b) a muscle specific control element; (c) an intron; (d) a polyadenylation signal sequence; and (c) a 3′-inverted terminal repeat (ITR). In an exemplary embodiment, the rAAV comprises an AAV capsid and a vector genome packaged therein, wherein the packaged vector genome comprises: (i) a micro-dystrophin coding sequence that is at least 99% (e.g., from 99% to 100%) identical to SEQ ID NO: 1; and optionally (ii) one or more additional sequence elements selected from: (a) a 5′-inverted terminal repeat (ITR); (b) a muscle specific control element; (c) an intron; (d) a polyadenylation signal sequence; and (e) a 3′-inverted terminal repeat (ITR). In a further exemplary embodiment, the rAAV comprises an AAV capsid and a vector genome packaged therein, wherein the packaged vector genome comprises: (i) a micro-dystrophin coding sequence set forth in SEQ ID NO: 1; and optionally (ii) one or more additional sequence elements selected from: (a) a 5′-inverted terminal repeat (ITR); (b) a muscle specific control element; (c) an intron; (d) a polyadenylation signal sequence; and (e) a 3′-inverted terminal repeat (ITR). In some embodiments, the AAV capsid is selected from an AAV of serotype 8, serotype 9, rh74, and hu37. In an exemplary embodiment, the AAV capsid is an hu37 (AAVhu37) capsid.

In another embodiment, the present disclosure provides a method of treating a muscular dystrophy (e.g., DMD) comprising administering an rAAV that comprises an AAV capsid and a vector genome packaged therein, wherein the packaged vector genome comprises a sequence which is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 3. In an exemplary embodiment, the rAAV comprises an AAV capsid and a packaged vector genome, wherein the packaged vector genome comprises a sequence which is at least 99% (e.g., from 99% to 100%) identical to SEQ ID NO: 3. In a further exemplary embodiment, the rAAV comprises an AAV capsid and a packaged vector genome, wherein the packaged vector genome comprises or consists of a sequence set forth in SEQ ID NO: 3. In some embodiments, the AAV capsid is selected from an AAV of serotype 8, serotype 9, rh74, and hu37. In an exemplary embodiment, the AAV capsid is an hu37 (AAVhu37) capsid.

In certain embodiments, the present disclosure provides a method of treating a muscular dystrophy (e.g., DMD) in a human subject diagnosed with at least one mutation in dystrophin, wherein said method comprises administering to the subject a therapeutically effective amount of at least one rAAV disclosed herein.

Both DMD and Becker muscular dystrophy (BMD) are caused by mutations in the dystrophin gene (also known as the DMD gene) on the X chromosome in the Xp21 region (MIM 300377), which spans 2.4 Mb of genomic DNA. The dystrophin gene is the largest human gene, containing 79 exons that encode a 14-Kb mRNA and produce a 427-kilo Dalton (kDa) membrane protein called dystrophin which is vital to the formation of a dystrophin-associated glycoprotein complex (DGC). Non-limiting lists of pathogenic mutations in dystrophin are described in Tuffery-Giruad et al., 2009, Hum Mutat 30 (6): 934-45, in Takeshima et al., 2010, J Hum Gen 55:379-88, in Bladen et al., 2015, Hum Mutat 36 (4): 395-402, and the TREAT-NMD DMD Global Database.

In certain embodiments, the present disclosure provides a method of treating a muscular dystrophy (e.g., DMD) in a human subject diagnosed with at least one mutation in dystrophin, wherein said method comprises administering an rAAV that comprises an AAV capsid and a vector genome packaged therein, wherein the packaged vector genome comprises: (i) a micro-dystrophin coding sequence that is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 1; and optionally (ii) one or more additional sequence elements selected from: (a) a 5′-inverted terminal repeat (ITR); (b) a muscle specific control element; (c) an intron; (d) a polyadenylation signal sequence; and (c) a 3′-inverted terminal repeat (ITR). In an exemplary embodiment, the rAAV comprises an AAV capsid and a vector genome packaged therein, wherein the packaged vector genome comprises: (i) a micro-dystrophin coding sequence that is at least 99% (e.g., from 99% to 100%) identical to SEQ ID NO: 1; and optionally (ii) one or more additional sequence elements selected from: (a) a 5′-inverted terminal repeat (ITR); (b) a muscle specific control element; (c) an intron; (d) a polyadenylation signal sequence; and (c) a 3′-inverted terminal repeat (ITR). In a further exemplary embodiment, the rAAV comprises an AAV capsid and a vector genome packaged therein, wherein the packaged vector genome comprises: (i) a micro-dystrophin coding sequence set forth in SEQ ID NO: 1; and optionally (ii) one or more additional sequence elements selected from: (a) a 5″-inverted terminal repeat (ITR); (b) a muscle specific control element; (c) an intron; (d) a polyadenylation signal sequence; and (e) a 3′-inverted terminal repeat (ITR). In some embodiments, the AAV capsid is selected from an AAV of serotype 8, serotype 9, rh74, and hu37. In an exemplary embodiment, the AAV capsid is an hu37 (AAVhu37) capsid.

In certain embodiments, the present disclosure provides a method of treating a muscular dystrophy (e.g., DMD) in a human subject diagnosed with at least one mutation in dystrophin, wherein said method comprises administering an rAAV that comprises an AAV capsid and a vector genome packaged therein, wherein the packaged vector genome comprises a sequence which is at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 3. In an exemplary embodiment, the rAAV comprises an AAV capsid and a packaged vector genome, wherein the packaged vector genome comprises a sequence which is at least 99% (e.g., from 99% to 100%) identical to SEQ ID NO: 3. In a further exemplary embodiment, the rAAV comprises an AAV capsid and a packaged vector genome, wherein the packaged vector genome comprises or consists of a sequence set forth in SEQ ID NO: 3. In some embodiments, the AAV capsid is selected from an AAV of serotype 8, serotype 9, rh74, and hu37. In an exemplary embodiment, the AAV capsid is an hu37 (AAVhu37) capsid.

Any suitable method or route can be used to administer a synthetic nucleic acid, an rAAV, a synthetic nucleic acid-containing pharmaceutical composition, or an rAAV-containing pharmaceutical composition described herein.

In specific embodiments where the pharmaceutical composition comprises an rAAV, the pharmaceutical composition may be administered via a variety of different routes, including, but not limited to, intravenously, subcutaneously, intramuscularly, intradermally, intraperitoneally, and intrathecally. In an exemplary embodiment, a pharmaceutical composition comprising an rAAV of the invention is administered intravenously or intramuscularly. In any of the uses described herein, the pharmaceutical composition comprising an rAAV of the invention may be administered locally or systemically. In some embodiments, the pharmaceutical composition comprising an rAAV of the invention may be administered via injection, infusion, or implantation.

The specific dose administered can be a uniform dose for each patient, for example, 1.0×10¹²-1.0×10¹⁶ genome copies (GC) of virus per patient. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can also be adjusted as the progress of the disease is monitored.

In some embodiments, the rAAV is administered at a dose of, e.g., about 1.0×10¹² genome copies per kilogram of patient body weight (GC/kg) to about 1×10¹⁶ GC/kg, about 1×10¹³ genome copies per kilogram of patient body weight (GC/kg) to about 1×10¹⁵ GC/kg, or about 5×10¹³ to about 5×10¹⁴ GC/kg, as measured by qPCR or digital droplet PCR (ddPCR). In some embodiments, the rAAV is administered at a dose at or about 1×10¹³ GC/kg, at or about 1.5×10¹³ GC/kg, at or about 2×10¹³ GC/kg, at or about 2.5×10¹³ GC/kg, at or about 3×10¹³ GC/kg, at or about 3.5×10¹³ GC/kg, at or about 4×10¹³ GC/kg, at or about 4.5×10¹³ GC/kg, at or about 5×10¹³ GC/kg, at or about 5.5×10¹³ GC/kg, at or about 6×10¹³ GC/kg, at or about 6.5×10¹³ GC/kg, at or about 7×10¹³ GC/kg, at or about 7.5×10¹³ GC/kg, at or about 8×10¹³ GC/kg, at or about 8.5×10¹³ GC/kg, at or about 9×10¹³ GC/kg, at or about 9.5×10¹³ GC/kg, at or about 1×10¹⁴ GC/kg, at or about 1.5×10¹⁴ GC/kg, at or about 2×10¹⁴ GC/kg, at or about 2.5×10¹⁴ GC/kg, at or about 3×10¹⁴ GC/kg, at or about 3.5×10¹⁴ GC/kg, at or about 4×10¹⁴ GC/kg, at or about 4.5×10¹⁴ GC/kg, at or about 5×10¹⁴ GC/kg, at or about 5.5×10¹⁴ GC/kg, at or about 6×10¹⁴ GC/kg, at or about 6.5×10¹⁴ GC/kg, at or about 7×10¹⁴ GC/kg, at or about 7.5×10¹⁴ GC/kg, at or about 8×10¹⁴ GC/kg, at or about 8.5×10¹⁴ GC/kg, at or about 9×10¹⁴ GC/kg, at or about 9.5×10¹⁴ GC/kg, or at or about 1×10¹⁵ GC/kg. The rAAV can be administered in a single dose, or in multiple doses (such as 2, 3, 4, 5, 6, 7, 8, 9, 10 or more doses) as needed for the desired therapeutic results.

In some embodiments, the methods of treating a muscular dystrophy (e.g. DMD) according to the instant invention may further comprise administration of an IgG-degrading protease prior to administration of an rAAV described herein. Accordingly, the present disclosure provides a method of treating a muscular dystrophy (e.g., DMD) comprising first administering an IgG-degrading protease and then subsequently administering an rAAV that includes an AAV capsid and a vector genome packaged therein, wherein the vector genome comprises a coding sequence for a human micro-dystrophin protein.

In some embodiments, a method of treating a muscular dystrophy (e.g., DMD) according to the instant invention is performed on a human subject who has been administered an IgG-degrading protease.

Examples of proteases that may be used in the instant invention include, for example and without limitation, those described in WO/2020/016318 and/or WO/2020/159970, including, for example, cysteine proteases from Streptococcus pyogenes, Streptococcus equi, Mycoplasma canis, Streptococcus agalactiae. Streptococcus pseudoporcinus, or Pseudomonas putida.

In certain embodiments, the IgG-degrading protease is the IdeS from Streptococcus pyogenes (SEQ ID NO: 13) or a protease which is at least 80%, at least 85%, at least 90%, at least 95%, at least 96%, at least 97%, at least 98%, or at least 99% identical to SEQ ID NO: 13. In some embodiments, the protease is an engineered variant of SEQ ID NO: 13. Examples of engineered IdeS proteases are described in WO/2020/016318 and U.S. Patent Publication Nos. 20180023070 and 20180037962. In some embodiments, the engineered IdeS variant may have 1, 2, 3, 4, 5, or more amino acid modifications relative to SEQ ID NO: 13.

In certain embodiments, the IgG-degrading protease is the IdeZ from Streptococcus equi (SEQ ID NO: 14) or a protease which is at least 80%, at least 85%, at least 90%, at least 95%, at least 96%, at least 97%, at least 98%, or at least 99% identical to SEQ ID NO: 14. In some embodiments, the protease is an engineered variant of SEQ ID NO: 14. Examples of engineered IdeZ proteases are described in WO/2020/016318. In some embodiments, the engineered IdeZ variant may have 1, 2, 3, 4, 5, or more amino acid modifications relative to SEQ ID NO: 14.

Other proteases that may be used in the instant invention include, for example and without limitation, IgdE enzymes from Streptococcus suis, Streptococcus porcinus, and Streptococcus equi, described in WO/2017/134274.

In some embodiments, the IgG-degrading protease may be encapsulated in or complexed with liposomes, nanoparticles, lipid nanoparticles (LNPs), polymers, microparticles, microcapsules, micelles, or extracellular vesicles.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the present disclosure, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within the present disclosure, embodiments have been described and depicted in a way that enables a clear and concise disclosure to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including” is intended merely to illustrate better the present invention and does not pose a limitation on the scope of the invention unless claimed. No language in the specification should be construed as indicating any non-claimed element as essential to the practice of the present invention.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1

The purpose of this example is to compare the protein expression of a native coding sequence for the MD5 micro-dystrophin (SEQ ID NO: 8) and a synthetic codon-optimized coding sequence (SEQ ID NO: 1) for the MD5 micro-dystrophin in an in vitro system.

In this example, C2C12 murine myoblasts were differentiated into myotubes for 3 days, followed by infection with rAAVs comprising an AAVhu37 capsid and a vector genome packaged therein encoding either a native coding sequence (SEQ ID NO: 8) or a synthetic codon-optimized coding sequence (SEQ ID NO: 1) for the MD5 micro-dystrophin at 1.8E7 vector genomes/cell. Cells were harvested 96 hours post-infection and cellular protein was isolated. Micro-dystrophin protein expression levels were determined via Meso Scale Discovery (MSD) ELISA utilizing a commercially available antibody specific for human dystrophin. Raw MSD ELISA reads were normalized to the native micro-dystrophin group mean value and expressed as fold change.

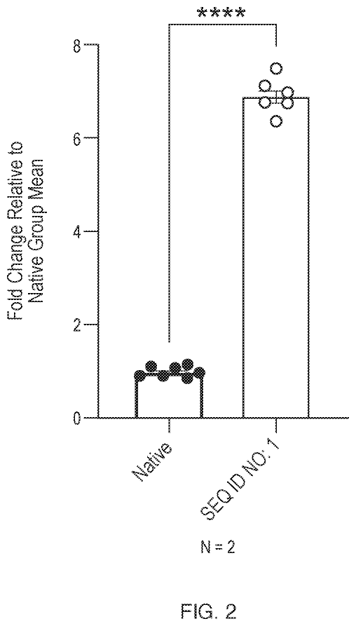

As shown in FIG. 2, the synthetic codon-optimized coding sequence (SEQ ID NO: 1) for micro-dystrophin demonstrated a statistically significant (p<0.0001), approximately 7-fold increase in protein expression compared to the native coding sequence for micro-dystrophin (n=2). Beyond the differences in coding sequences, all other components of the rAAV were identical, suggesting that the use of the synthetic codon-optimized coding sequence of SEQ ID NO: 1 was responsible for improvements observed in protein expression.

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

### Sequence Listing

SEQ ID NO: 1 (Synthetic Nucleic Acid Encoding MD)
ATGCTGTGGTGGGAGGAAGTGGAAGATTGCTACGAGCGCGAGGACGTGCAGAAGA
AAACCTTCACCAAATGGGTCAACGCCCAGTTCAGCAAGTTCGGCAAGCAGCACATC
GAGAACCTGTTCAGCGACCTGCAGGACGGCAGACGGCTGCTGGATCTGCTGGAAGG
CCTGACCGGACAGAAGCTGCCCAAAGAGAAGGGCAGCACCAGAGTGCACGCCCTG
AACAACGTGAACAAGGCCCTGCGGGTGCTGCAGAACAACAATGTGGACCTGGTGAA
CATTGGCAGCACAGACATTGTGGATGGCAACCACAAGCTGACCCTGGGCCTGATCT
GGAACATCATCCTGCACTGGCAAGTGAAGAACGTGATGAAGAACATCATGGCCGGC
CTGCAGCAGACCAACAGCGAGAAGATCCTGCTGAGCTGGGTGCGCCAGAGCACCAG
AAACTACCCCCAAGTGAACGTGATCAACTTCACCACCTCTTGGAGCGACGGCCTGG
CCCTGAATGCCCTGATCCACAGCCACAGACCTGACCTGTTTGACTGGAACAGTGTTG
TGTGTCAGCAGAGTGCCACCCAGAGGCTGGAACACGCCTTCAATATCGCCAGATAC
CAGCTGGGCATCGAGAAGCTGCTGGACCCCGAGGATGTGGACACCACCTACCCCGA
CAAGAAATCCATCCTGATGTATATCACCAGCCTGTTCCAGGTGCTGCCTCAGCAGGT
GTCCATCGAGGCCATCCAGGAAGTGGAAATGCTGCCCAGACCCCCCAAAGTGACCA
AAGAGGAACACTTCCAGCTGCACCACCAGATGCACTACTCTCAGCAGATCACCGTG
TCCCTGGCCCAGGGCTACGAGAGAACCAGCAGCCCCAAGCCCCGGTTCAAGAGCTA
CGCCTATACCCAGGCCGCCTACGTGACCACCAGCGACCCTACCAGAAGCCCATTCCC
CAGCCAGCATCTGGAAGCCCCCGAGGACAAGAGCTTCGGCAGCAGCCTGATGGAAA
GCGAAGTGAACCTGGATAGATACCAGACCGCCCTGGAAGAGGTGCTGTCCTGGCTG
CTGAGCGCCGAGGATACACTGCAGGCTCAGGGCGAGATCAGCAACGACGTGGAAGT
CGTGAAGGACCAGTTCCACACCCACGAGGGCTACATGATGGACCTGACAGCCCACC
AGGGCAGAGTGGGCAACATTCTGCAGCTGGGCTCCAAGCTGATCGGCACCGGCAAG
CTGAGCGAGGACGAAGAGACAGAGGTGCAGGAACAGATGAACCTGCTGAACAGCA
GATGGGAGTGCCTGAGAGTGGCCAGCATGGAAAAGCAGAGCAACCTGCACAGCTA
CGTGCCCAGCACCTACCTGACCGAGATCACCCATGTGTCCCAGGCCCTGCTGGAAGT
GGAACAGCTGCTGAACGCCCCCGATCTGTGCGCCAAGGACTTCGAGGATCTGTTCA
AGCAGGAAGAGAGCCTGAAGAATATCAAGGACTCTCTGCAGCAGTCCAGCGGCAG
AATCGACATCATCCACAGCAAGAAAACAGCCGCCCTGCAGTCCGCCACCCCCGTGG
AAAGAGTGAAGCTGCAGGAAGCCCTGTCCCAGCTGGACTTCCAGTGGGAGAAAGTG
AACAAGATGTACAAGGACAGGCAGGGCAGATTTGACAGGTCTGTGGAAAAGTGGA
GGAGGTTCCACTACGACATCAAGATCTTCAACCAGTGGCTGACCGAGGCCGAGCAG
TTCCTGAGAAAGACCCAGATCCCCGAGAACTGGGAGCACGCCAAGTACAAGTGGTA
TCTGAAAGAACTGCAGGATGGCATCGGCCAGAGACAGACAGTTGTGAGGACACTGA
ATGCCACAGGAGAGGAAATCATCCAGCAGAGCAGCAAGACAGATGCCAGTATTCTG
CAGGAAAAGCTGGGCAGCCTGAACCTGAGATGGCAGGAAGTGTGCAAGCAGCTGTC
CGACCGGAAGAAGAGACTGGAAGAACAGAGCGACCAGTGGAAGCGGCTGCATCTG
TCACTGCAGGAACTGCTCGTGTGGCTGCAGCTGAAGGATGATGAGCTGAGCAGACA
GGCCCCTATTGGAGGAGATTTTCCTGCTGTGCAGAAACAGAATGATGTGCACCGGG
CCTTCAAGAGAGAGCTGAAAAAAAAGAACCCGTGATCATGAGCACCCTGGAAACC
GTGCGGATCTTTCTGACCGAGCAGCCCCTGGAAGGACTGGAAAAACTGTACCAGGA
ACCCAGAGAGCTGCCCCCTGAAGAACGGGCCCAGAACGTGACCAGACTGCTGCGGA
AGCAGGCCGAGGAAGTGAACACCGAATGGGAGAAGCTGAACCTGCACTCTGCTGAC
TGGCAGAGGAAGATTGATGAGACACTGGAACGGCTGCAGGAACTGCAGGAGGCCA
CCGACGAGCTGGACCTGAAACTGAGACAGGCCGAAGTGATCAAGGGCAGCTGGCA
GCCAGTGGGCGACCTGCTGATCGACAGCCTGCAGGATCACCTGGAAAAAGTGAAAG
CCCTGAGAGGCGAGATCGCCCCCCTGAAAGAAAACGTGTCCCACGTGAACGACCTG
GCCCGGCAGCTGACAACACTGGGCATCCAGCTGAGCCCCTACAACCTGTCCACACT
GGAAGATCTGAACACCCGGTGGAAACTGCTGCAGGTGGCCGTGGAAGATAGAGTGC
GGCAGCTGCACGAGGCCCACAGAGATTTTGGCCCTGCCTCCCAGCACTTCCTGAGCA
CATCTGTGCAGGGCCCCTGGGAGAGAGCCATCTCCCCCAACAAGGTGCCCTACTAC
ATCAACCACGAGACACAGACCACCTGTTGGGACCACCCCAAGATGACCGAGCTGTA
CCAGAGCCTGGCCGACCTGAACAATGTGAGGTTCAGTGCCTACAGGACAGCCATGA
AGCTGAGGAGACTGCAGAAAGCTCTGTGCCTGGACCTGCTGTCCCTGTCCGCCGCTT
GTGATGCCCTGGACCAGCACAACCTGAAGCAGAACGACCAGCCCATGGATATCCTG
CAGATCATCAACTGCCTGACCACCATCTACGACCGCCTGGAACAGGAACACAACAA
CCTCGTGAATGTGCCCCTGTGCGTGGACATGTGCCTGAATTGGCTGCTGAATGTGTA
TGACACAGGCAGGACAGGCAGGATCAGAGTGCTGAGCTTCAAGACCGGCATCATCA
GCCTGTGCAAGGCCCACCTGGAAGATAAGTACCGCTACCTGTTCAAACAGGTGGCC
AGCTCCACCGGCTTTTGCGACCAGAGAAGGCTGGGCCTGCTGCTGCACGACAGCAT
CCAGATCCCTAGACAGCTGGGCGAGGTGGCCTCTTTTGGAGGCAGCAATATTGAGC
CTAGTGTGAGGAGCTGCTTTCAGTTCGCCAACAACAAGCCTGAGATTGAGGCTGCCC
TGTTCCTGGACTGGATGCGGCTGGAACCCCAGAGCATGGTGTGGCTGCCTGTGCTGC
ATAGAGTGGCCGCTGCCGAGACAGCCAAGCACCAGGCCAAGTGCAACATCTGCAAA
GAGTGCCCCATCATCGGCTTCCGGTACAGAAGCCTGAAGCACTTCAACTACGATATC
TGCCAGAGCTGCTTTTTCAGCGGACGGGTGGCCAAGGGCCACAAAATGCACTACCC
CATGGTGGAATACTGCACCCCCACCACCTCCGGGGAGGATGTGCGGGATTTTGCCA
AGGTGCTGAAAAACAAGTTCCGGACCAAGCGCTACTTCGCCAAACACCCCCGGATG
GGCTATCTGCCCGTGCAGACAGTGCTGGAAGGCGACAACATGGAAACCGACACCAT
G
SEQ ID NO: 2 (Synthetic Nucleic Acid Encoding MD + STOP)
ATGCTGTGGTGGGAGGAAGTGGAAGATTGCTACGAGCGCGAGGACGTGCAGAAGA
AAACCTTCACCAAATGGGTCAACGCCCAGTTCAGCAAGTTCGGCAAGCAGCACATC
GAGAACCTGTTCAGCGACCTGCAGGACGGCAGACGGCTGCTGGATCTGCTGGAAGG
CCTGACCGGACAGAAGCTGCCCAAAGAGAAGGGCAGCACCAGAGTGCACGCCCTG
AACAACGTGAACAAGGCCCTGCGGGTGCTGCAGAACAACAATGTGGACCTGGTGAA
CATTGGCAGCACAGACATTGTGGATGGCAACCACAAGCTGACCCTGGGCCTGATCT
GGAACATCATCCTGCACTGGCAAGTGAAGAACGTGATGAAGAACATCATGGCCGGC
CTGCAGCAGACCAACAGCGAGAAGATCCTGCTGAGCTGGGTGCGCCAGAGCACCAG
AAACTACCCCCAAGTGAACGTGATCAACTTCACCACCTCTTGGAGCGACGGCCTGG
CCCTGAATGCCCTGATCCACAGCCACAGACCTGACCTGTTTGACTGGAACAGTGTTG
TGTGTCAGCAGAGTGCCACCCAGAGGCTGGAACACGCCTTCAATATCGCCAGATAC
CAGCTGGGCATCGAGAAGCTGCTGGACCCCGAGGATGTGGACACCACCTACCCCGA
CAAGAAATCCATCCTGATGTATATCACCAGCCTGTTCCAGGTGCTGCCTCAGCAGGT
GTCCATCGAGGCCATCCAGGAAGTGGAAATGCTGCCCAGACCCCCCAAAGTGACCA
AAGAGGAACACTTCCAGCTGCACCACCAGATGCACTACTCTCAGCAGATCACCGTG
TCCCTGGCCCAGGGCTACGAGAGAACCAGCAGCCCCAAGCCCCGGTTCAAGAGCTA
CGCCTATACCCAGGCCGCCTACGTGACCACCAGCGACCCTACCAGAAGCCCATTCCC
CAGCCAGCATCTGGAAGCCCCCGAGGACAAGAGCTTCGGCAGCAGCCTGATGGAAA
GCGAAGTGAACCTGGATAGATACCAGACCGCCCTGGAAGAGGTGCTGTCCTGGCTG
CTGAGCGCCGAGGATACACTGCAGGCTCAGGGCGAGATCAGCAACGACGTGGAAGT
CGTGAAGGACCAGTTCCACACCCACGAGGGCTACATGATGGACCTGACAGCCCACC
AGGGCAGAGTGGGCAACATTCTGCAGCTGGGCTCCAAGCTGATCGGCACCGGCAAG
CTGAGCGAGGACGAAGAGACAGAGGTGCAGGAACAGATGAACCTGCTGAACAGCA
GATGGGAGTGCCTGAGAGTGGCCAGCATGGAAAAGCAGAGCAACCTGCACAGCTA
CGTGCCCAGCACCTACCTGACCGAGATCACCCATGTGTCCCAGGCCCTGCTGGAAGT
GGAACAGCTGCTGAACGCCCCCGATCTGTGCGCCAAGGACTTCGAGGATCTGTTCA
AGCAGGAAGAGAGCCTGAAGAATATCAAGGACTCTCTGCAGCAGTCCAGCGGCAG
AATCGACATCATCCACAGCAAGAAAACAGCCGCCCTGCAGTCCGCCACCCCCGTGG
AAAGAGTGAAGCTGCAGGAAGCCCTGTCCCAGCTGGACTTCCAGTGGGAGAAAGTG
AACAAGATGTACAAGGACAGGCAGGGCAGATTTGACAGGTCTGTGGAAAAGTGGA
GGAGGTTCCACTACGACATCAAGATCTTCAACCAGTGGCTGACCGAGGCCGAGCAG
TTCCTGAGAAAGACCCAGATCCCCGAGAACTGGGAGCACGCCAAGTACAAGTGGTA
TCTGAAAGAACTGCAGGATGGCATCGGCCAGAGACAGACAGTTGTGAGGACACTGA
ATGCCACAGGAGAGGAAATCATCCAGCAGAGCAGCAAGACAGATGCCAGTATTCTG
CAGGAAAAGCTGGGCAGCCTGAACCTGAGATGGCAGGAAGTGTGCAAGCAGCTGTC
CGACCGGAAGAAGAGACTGGAAGAACAGAGCGACCAGTGGAAGCGGCTGCATCTG
TCACTGCAGGAACTGCTCGTGTGGCTGCAGCTGAAGGATGATGAGCTGAGCAGACA
GGCCCCTATTGGAGGAGATTTTCCTGCTGTGCAGAAACAGAATGATGTGCACCGGG
CCTTCAAGAGAGAGCTGAAAACAAAAGAACCCGTGATCATGAGCACCCTGGAAACC
GTGCGGATCTTTCTGACCGAGCAGCCCCTGGAAGGACTGGAAAAACTGTACCAGGA
ACCCAGAGAGCTGCCCCCTGAAGAACGGGCCCAGAACGTGACCAGACTGCTGCGGA
AGCAGGCCGAGGAAGTGAACACCGAATGGGAGAAGCTGAACCTGCACTCTGCTGAC
TGGCAGAGGAAGATTGATGAGACACTGGAACGGCTGCAGGAACTGCAGGAGGCCA
CCGACGAGCTGGACCTGAAACTGAGACAGGCCGAAGTGATCAAGGGCAGCTGGCA
GCCAGTGGGCGACCTGCTGATCGACAGCCTGCAGGATCACCTGGAAAAAGTGAAAG
CCCTGAGAGGCGAGATCGCCCCCCTGAAAGAAAACGTGTCCCACGTGAACGACCTG
GCCCGGCAGCTGACAACACTGGGCATCCAGCTGAGCCCCTACAACCTGTCCACACT
GGAAGATCTGAACACCCGGTGGAAACTGCTGCAGGTGGCCGTGGAAGATAGAGTGC
GGCAGCTGCACGAGGCCCACAGAGATTTTGGCCCTGCCTOCCAGCACTTCCTGAGCA
CATCTGTGCAGGGCCCCTGGGAGAGAGCCATCTCCCCCAACAAGGTGCCCTACTAC
ATCAACCACGAGACACAGACCACCTGTTGGGACCACCCCAAGATGACCGAGCTGTA
CCAGAGCCTGGCCGACCTGAACAATGTGAGGTTCAGTGCCTACAGGACAGCCATGA
AGCTGAGGAGACTGCAGAAAGCTCTGTGCCTGGACCTGCTGTCCCTGTCCGCCGCTT
GTGATGCCCTGGACCAGCACAACCTGAAGCAGAACGACCAGCCCATGGATATCCTG
CAGATCATCAACTGCCTGACCACCATCTACGACCGCCTGGAACAGGAACACAACAA
CCTCGTGAATGTGCCCCTGTGCGTGGACATGTGCCTGAATTGGCTGCTGAATGTGTA
TGACACAGGCAGGACAGGCAGGATCAGAGTGCTGAGCTTCAAGACCGGCATCATCA
GCCTGTGCAAGGCCCACCTGGAAGATAAGTACCGCTACCTGTTCAAACAGGTGGCC
AGCTCCACCGGCTTTTGCGACCAGAGAAGGCTGGGCCTGCTGCTGCACGACAGCAT
CCAGATCCCTAGACAGCTGGGCGAGGTGGCCTCTTTTGGAGGCAGCAATATTGAGC
CTAGTGTGAGGAGCTGCTTTCAGTTCGCCAACAACAAGCCTGAGATTGAGGCTGCCC
TGTTCCTGGACTGGATGCGGCTGGAACCCCAGAGCATGGTGTGGCTGCCTGTGCTGC
ATAGAGTGGCCGCTGCCGAGACAGCCAAGCACCAGGCCAAGTGCAACATCTGCAAA
GAGTGCCCCATCATCGGCTTCCGGTACAGAAGCCTGAAGCACTTCAACTACGATATC
TGCCAGAGCTGCTTTTTCAGCGGACGGGTGGCCAAGGGCCACAAAATGCACTACCC
CATGGTGGAATACTGCACCCCCACCACCTCCGGGGAGGATGTGCGGGATTTTGCCA
AGGTGCTGAAAAACAAGTTCCGGACCAAGCGCTACTTCGCCAAACACCCCCGGATG
GGCTATCTGCCCGTGCAGACAGTGCTGGAAGGCGACAACATGGAAACCGACACCAT
GTAG
SEQ ID NO: 3 (DTC630 Full Genome)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTCAGATCCAAGCTTTAGACTAGCATG
CTGCCCATGTAAGGAGGCAAGGCCTGGGGACACCCGAGATGCCTGGTTATAATTAA
CCCAGACATGTGGCTGCCCCCCCCCCCCCAACACCTGCTGCCTCTAAAAATAACCCT
GCATGCCATGTTCCCGGCGAAGGGCCAGCTGTCCCCCGCCAGCTAGACTCAGCACTT
AGTTTAGGAACCAGTGAGCAAGTCAGCCCTTGGGGCAGCCCATACAAGGCCATGGG
GCTGGGCAAGCTGCACGCCTGGGTCCGGGGTGGGCACGGTGCCCGGGCAACGAGCT
GAAAGCTCATCTGCTCTCAGGGGCCCCTCCCTGGGGACAGCCCCTCCTGGCTAGTCA
CACCCTGTAGGCTCCTCTATATAACCCAGGGGCACAGGGGCTGCCCTCATTCTACCA
CCACCTCCACAGCACAGACAGACACTCAGGAGCCAGCCAAAACTAGTGTATCAAGG
TTACAAGACAGGTTTAAGGAGACCAATAGAAACTGGGCTTGTCGAGACAGAGAAGA
CTCTTGCGTTTCTGATAGGCACCTATTGGTCTTACTGACATCCACTTTGCCTTTCTCT
CCACAGGCCTCTAGAACCATGCTGTGGTGGGAGGAAGTGGAAGATTGCTACGAGCG
CGAGGACGTGCAGAAGAAAACCTTCACCAAATGGGTCAACGCCCAGTTCAGCAAGT
TCGGCAAGCAGCACATCGAGAACCTGTTCAGCGACCTGCAGGACGGCAGACGGCTG
CTGGATCTGCTGGAAGGCCTGACCGGACAGAAGCTGCCCAAAGAGAAGGGCAGCA
CCAGAGTGCACGCCCTGAACAACGTGAACAAGGCCCTGCGGGTGCTGCAGAACAAC
AATGTGGACCTGGTGAACATTGGCAGCACAGACATTGTGGATGGCAACCACAAGCT
GACCCTGGGCCTGATCTGGAACATCATCCTGCACTGGCAAGTGAAGAACGTGATGA
AGAACATCATGGCCGGCCTGCAGCAGACCAACAGCGAGAAGATCCTGCTGAGCTGG
GTGCGCCAGAGCACCAGAAACTACCCCCAAGTGAACGTGATCAACTTCACCACCTC
TTGGAGCGACGGCCTGGCCCTGAATGCCCTGATCCACAGCCACAGACCTGACCTGTT
TGACTGGAACAGTGTTGTGTGTCAGCAGAGTGCCACCCAGAGGCTGGAACACGCCT
TCAATATCGCCAGATACCAGCTGGGCATCGAGAAGCTGCTGGACCCCGAGGATGTG
GACACCACCTACCCCGACAAGAAATCCATCCTGATGTATATCACCAGCCTGTTCCAG
GTGCTGCCTCAGCAGGTGTCCATCGAGGCCATCCAGGAAGTGGAAATGCTGCCCAG
ACCCCCCAAAGTGACCAAAGAGGAACACTTCCAGCTGCACCACCAGATGCACTACT
CTCAGCAGATCACCGTGTCCCTGGCCCAGGGCTACGAGAGAACCAGCAGCCCCAAG
CCCCGGTTCAAGAGCTACGCCTATACCCAGGCCGCCTACGTGACCACCAGCGACCCT
ACCAGAAGCCCATTCCCCAGCCAGCATCTGGAAGCCCCCGAGGACAAGAGCTTCGG
CAGCAGCCTGATGGAAAGCGAAGTGAACCTGGATAGATACCAGACCGCCCTGGAAG
AGGTGCTGTCCTGGCTGCTGAGCGCCGAGGATACACTGCAGGCTCAGGGCGAGATC
AGCAACGACGTGGAAGTCGTGAAGGACCAGTTCCACACCCACGAGGGCTACATGAT
GGACCTGACAGCCCACCAGGGCAGAGTGGGCAACATTCTGCAGCTGGGCTCCAAGC
TGATCGGCACCGGCAAGCTGAGCGAGGACGAAGAGACAGAGGTGCAGGAACAGAT
GAACCTGCTGAACAGCAGATGGGAGTGCCTGAGAGTGGCCAGCATGGAAAAGCAG
AGCAACCTGCACAGCTACGTGCCCAGCACCTACCTGACCGAGATCACCCATGTGTCC
CAGGCCCTGCTGGAAGTGGAACAGCTGCTGAACGCCCCCGATCTGTGCGCCAAGGA
CTTCGAGGATCTGTTCAAGCAGGAAGAGAGCCTGAAGAATATCAAGGACTCTCTGC
AGCAGTCCAGCGGCAGAATCGACATCATCCACAGCAAGAAAACAGCCGCCCTGCAG
TCCGCCACCCCCGTGGAAAGAGTGAAGCTGCAGGAAGCCCTGTCCCAGCTGGACTT
CCAGTGGGAGAAAGTGAACAAGATGTACAAGGACAGGCAGGGCAGATTTGACAGG
TCTGTGGAAAAGTGGAGGAGGTTCCACTACGACATCAAGATCTTCAACCAGTGGCT
GACCGAGGCCGAGCAGTTCCTGAGAAAGACCCAGATCCCCGAGAACTGGGAGCAC
GCCAAGTACAAGTGGTATCTGAAAGAACTGCAGGATGGCATCGGCCAGAGACAGAC
AGTTGTGAGGACACTGAATGCCACAGGAGAGGAAATCATCCAGCAGAGCAGCAAG
ACAGATGCCAGTATTCTGCAGGAAAAGCTGGGCAGCCTGAACCTGAGATGGCAGGA
AGTGTGCAAGCAGCTGTCCGACCGGAAGAAGAGACTGGAAGAACAGAGCGACCAG
TGGAAGCGGCTGCATCTGTCACTGCAGGAACTGCTCGTGTGGCTGCAGCTGAAGGA
TGATGAGCTGAGCAGACAGGCCCCTATTGGAGGAGATTTTCCTGCTGTGCAGAAAC
AGAATGATGTGCACCGGGCCTTCAAGAGAGAGCTGAAAACAAAAGAACCCGTGATC
ATGAGCACCCTGGAAACCGTGCGGATCTTTCTGACCGAGCAGCCCCTGGAAGGACT
GGAAAAACTGTACCAGGAACCCAGAGAGCTGCCCCCTGAAGAACGGGCCCAGAAC
GTGACCAGACTGCTGCGGAAGCAGGCCGAGGAAGTGAACACCGAATGGGAGAAGC
TGAACCTGCACTCTGCTGACTGGCAGAGGAAGATTGATGAGACACTGGAACGGCTG
CAGGAACTGCAGGAGGCCACCGACGAGCTGGACCTGAAACTGAGACAGGCCGAAG
TGATCAAGGGCAGCTGGCAGCCAGTGGGCGACCTGCTGATCGACAGCCTGCAGGAT
CACCTGGAAAAAGTGAAAGCCCTGAGAGGCGAGATCGCCCCCCTGAAAGAAAACG
TGTCCCACGTGAACGACCTGGCCCGGCAGCTGACAACACTGGGCATCCAGCTGAGC
CCCTACAACCTGTCCACACTGGAAGATCTGAACACCCGGTGGAAACTGCTGCAGGT
GGCCGTGGAAGATAGAGTGCGGCAGCTGCACGAGGCCCACAGAGATTTTGGCCCTG
CCTCCCAGCACTTCCTGAGCACATCTGTGCAGGGCCCCTGGGAGAGAGCCATCTCCC
CCAACAAGGTGCCCTACTACATCAACCACGAGACACAGACCACCTGTTGGGACCAC
CCCAAGATGACCGAGCTGTACCAGAGCCTGGCCGACCTGAACAATGTGAGGTTCAG
TGCCTACAGGACAGCCATGAAGCTGAGGAGACTGCAGAAAGCTCTGTGCCTGGACC
TGCTGTCCCTGTCCGCCGCTTGTGATGCCCTGGACCAGCACAACCTGAAGCAGAACG
ACCAGCCCATGGATATCCTGCAGATCATCAACTGCCTGACCACCATCTACGACCGCC
TGGAACAGGAACACAACAACCTCGTGAATGTGCCCCTGTGCGTGGACATGTGCCTG
AATTGGCTGCTGAATGTGTATGACACAGGCAGGACAGGCAGGATCAGAGTGCTGAG
CTTCAAGACCGGCATCATCAGCCTGTGCAAGGCCCACCTGGAAGATAAGTACCGCT
ACCTGTTCAAACAGGTGGCCAGCTCCACCGGCTTTTGCGACCAGAGAAGGCTGGGC
CTGCTGCTGCACGACAGCATCCAGATCCCTAGACAGCTGGGCGAGGTGGCCTCTTTT
GGAGGCAGCAATATTGAGCCTAGTGTGAGGAGCTGCTTTCAGTTCGCCAACAACAA
GCCTGAGATTGAGGCTGCCCTGTTCCTGGACTGGATGCGGCTGGAACCCCAGAGCA
TGGTGTGGCTGCCTGTGCTGCATAGAGTGGCCGCTGCCGAGACAGCCAAGCACCAG
GCCAAGTGCAACATCTGCAAAGAGTGCCCCATCATCGGCTTCCGGTACAGAAGCCT
GAAGCACTTCAACTACGATATCTGCCAGAGCTGCTTTTTCAGCGGACGGGTGGCCAA
GGGCCACAAAATGCACTACCCCATGGTGGAATACTGCACCCCCACCACCTCCGGGG
AGGATGTGCGGGATTTTGCCAAGGTGCTGAAAAACAAGTTCCGGACCAAGCGCTAC
TTCGCCAAACACCCCCGGATGGGCTATCTGCCCGTGCAGACAGTGCTGGAAGGCGA
CAACATGGAAACCGACACCATGTAGGAAGTCTTTTAATAAAAGATCCTTATTTTCAT
TGGATCTGTGTGTTGGTTTTTTGTGTGCTCGAGGGATCTGAGGAACCCCTAGTGATG
GAGTTGGCCACTCCCTCTCTGCGCGCTCGCTCGCTCACTGAGGCCGCCCGGGCAAAG
CCCGGGCGTCGGGCGACCTTTGGTCGCCCGGCCTCAGTGAGCGAGCGAGCGCGCAG
AGAGGGAGTGGCCAA
SEQ ID NO: 4 (AAV2 ITR)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCT
SEQ ID NO: 5 (AAV2 ITR)
AGGAACCCCTAGTGATGGAGTTGGCCACTCCCTCTCTGCGCGCTCGCTCGCTCACTG
AGGCCGCCCGGGCAAAGCCCGGGCGTCGGGCGACCTTTGGTCGCCCGGCCTCAGTG
AGCGAGCGAGCGCGCAGAGAGGGAGTGGCCAA
SEQ ID NO: 6 (CK8 PROMOTER)
TAGACTAGCATGCTGCCCATGTAAGGAGGCAAGGCCTGGGGACACCCGAGATGCCT
GGTTATAATTAACCCAGACATGTGGCTGCCCCCCCCCCCCCAACACCTGCTGCCTCT
AAAAATAACCCTGCATGCCATGTTCCCGGCGAAGGGCCAGCTGTCCCCCGCCAGCT
AGACTCAGCACTTAGTTTAGGAACCAGTGAGCAAGTCAGCCCTTGGGGCAGCCCAT
ACAAGGCCATGGGGCTGGGCAAGCTGCACGCCTGGGTCCGGGGTGGGCACGGTGCC
CGGGCAACGAGCTGAAAGCTCATCTGCTCTCAGGGGCCCCTCCCTGGGGACAGCCC
CTCCTGGCTAGTCACACCCTGTAGGCTCCTCTATATAACCCAGGGGCACAGGGGCTG
CCCTCATTCTACCACCACCTCCACAGCACAGACAGACACTCAGGAGCCAGCCA
SEQ ID NO: 7 (MHCK7 PROMOTER/ENHANCER)
GTTTAAACAAGCTTGCATGTCTAAGCTAGACCCTTCAGATTAAAAATAACTGAGGTA
AGGGCCTGGGTAGGGGAGGTGGTGTGAGACGCTCCTGTCTCTCCTCTATCTGCCCAT
CGGCCCTTTGGGGAGGAGGAATGTGCCCAAGGACTAAAAAAAGGCCATGGAGCCA
GAGGGGCGAGGGCAACAGACCTTTCATGGGCAAACCTTGGGGCCCTGCTGTCTAGC
ATGCCCCACTACGGGTCTAGGCTGCCCATGTAAGGAGGCAAGGCCTGGGGACACCC
GAGATGCCTGGTTATAATTAACCCAGACATGTGGCTGCCCCCCCCCCCCCAACACCT
GCTGCCTCTAAAAATAACCCTGTCCCTGGTGGATCCCCTGCATGCGAAGATCTTCGA
ACAAGGCTGTGGGGGACTGAGGGCAGGCTGTAACAGGCTTGGGGGCCAGGGCTTAT
ACGTGCCTGGGACTCCCAAAGTATTACTGTTCCATGTTCCCGGCGAAGGGCCAGCTG
TCCCCCGCCAGCTAGACTCAGCACTTAGTTTAGGAACCAGTGAGCAAGTCAGCCCTT
GGGGCAGCCCATACAAGGCCATGGGGCTGGGCAAGCTGCACGCCTGGGTCCGGGGT
GGGCACGGTGCCCGGGCAACGAGCTGAAAGCTCATCTGCTCTCAGGGGCCCCTCCC
TGGGGACAGCCCCTCCTGGCTAGTCACACCCTGTAGGCTCCTCTATATAACCCAGGG
GCACAGGGGCTGCCCTCATTCTACCACCACCTCCACAGCACAGACAGACACTCAGG
AGCCAGCCAGCGGCGCGCCC
SEQ ID NO: 8 (NATIVE MD5 CODING SEQUENCE)
ATGCTTTGGTGGGAAGAAGTAGAGGACTGTTATGAAAGAGAAGATGTTCAAAAGAA
AACATTCACAAAATGGGTAAATGCACAATTTTCTAAGTTTGGGAAGCAGCATATTG
AGAACCTCTTCAGTGACCTACAGGATGGGAGGCGCCTCCTAGACCTCCTCGAAGGC
CTGACAGGGCAAAAACTGCCAAAAGAAAAAGGATCCACAAGAGTTCATGCCCTGA
ACAATGTCAACAAGGCACTGCGGGTTTTGCAGAACAATAATGTTGATTTAGTGAAT
ATTGGAAGTACTGACATCGTAGATGGAAATCATAAACTGACTCTTGGTTTGATTTGG
AATATAATCCTCCACTGGCAGGTCAAAAATGTAATGAAAAATATCATGGCTGGATT
GCAACAAACCAACAGTGAAAAGATTCTCCTGAGCTGGGTCCGACAATCAACTCGTA
ATTATCCACAGGTTAATGTAATCAACTTCACCACCAGCTGGTCTGATGGCCTGGCTT
TGAATGCTCTCATCCATAGTCATAGGCCAGACCTATTTGACTGGAATAGTGTGGTTT
GCCAGCAGTCAGCCACACAACGACTGGAACATGCATTCAACATCGCCAGATATCAA
TTAGGCATAGAGAAACTACTCGATCCTGAAGATGTTGATACCACCTATCCAGATAA
GAAGTCCATCTTAATGTACATCACATCACTCTTCCAAGTTTTGCCTCAACAAGTGAG
CATTGAAGCCATCCAGGAAGTGGAAATGTTGCCAAGGCCACCTAAAGTGACTAAAG
AAGAACATTTTCAGTTACATCATCAAATGCACTATTCTCAACAGATCACGGTCAGTC
TAGCACAGGGATATGAGAGAACTTCTTCCCCTAAGCCTCGATTCAAGAGCTATGCCT
ACACACAGGCTGCTTATGTCACCACCTCTGACCCTACACGGAGCCCATTTCCTTCAC
AGCATTTGGAAGCTCCTGAAGACAAGTCATTTGGCAGTTCATTGATGGAGAGTGAA
GTAAACCTGGACCGTTATCAAACAGCTTTAGAAGAAGTATTATCGTGGCTTCTTTCT
GCTGAGGACACATTGCAAGCACAAGGAGAGATTTCTAATGATGTGGAAGTGGTGAA
AGACCAGTTTCATACTCATGAGGGGTACATGATGGATTTGACAGCCCATCAGGGCC
GGGTTGGTAATATTCTACAATTGGGAAGTAAGCTGATTGGAACAGGAAAATTATCA
GAAGATGAAGAAACTGAAGTACAAGAGCAGATGAATCTCCTAAATTCAAGATGGG
AATGCCTCAGGGTAGCTAGCATGGAAAAACAAAGCAATTTACATTCTTATGTGCCTT
CTACTTATTTGACTGAAATCACTCATGTCTCACAAGCCCTATTAGAAGTGGAACAAC
TTCTCAATGCTCCTGACCTCTGTGCTAAGGACTTTGAAGATCTCTTTAAGCAAGAGG
AGTCTCTGAAGAATATAAAAGATAGTCTACAACAAAGCTCAGGTCGGATTGACATT
ATTCATAGCAAGAAGACAGCAGCATTGCAAAGTGCAACGCCTGTGGAAAGGGTGAA
GCTACAGGAAGCTCTCTCCCAGCTTGATTTCCAATGGGAAAAAGTTAACAAAATGT
ACAAGGACCGACAAGGGCGATTTGACAGATCTGTTGAGAAATGGCGGCGTTTTCAT
TATGATATAAAGATATTTAATCAGTGGCTAACAGAAGCTGAACAGTTTCTCAGAAA
GACACAAATTCCTGAGAATTGGGAACATGCTAAATACAAATGGTATCTTAAGGAAC
TCCAGGATGGCATTGGGCAGCGGCAAACTGTTGTCAGAACATTGAATGCAACTGGG
GAAGAAATAATTCAGCAATCCTCAAAAACAGATGCCAGTATTCTACAGGAAAAATT
GGGAAGCCTGAATCTGCGGTGGCAGGAGGTCTGCAAACAGCTGTCAGACAGAAAA
AAGAGGCTAGAAGAACAATCTGACCAGTGGAAGCGTCTGCACCTTTCTCTGCAGGA
ACTTCTGGTGTGGCTACAGCTGAAAGATGATGAATTAAGCCGGCAGGCACCTATTG
GAGGCGACTTTCCAGCAGTTCAGAAGCAGAACGATGTACATAGGGCCTTCAAGAGG
GAATTGAAAACTAAAGAACCTGTAATCATGAGTACTCTTGAGACTGTACGAATATTT
CTGACAGAGCAGCCTTTGGAAGGACTAGAGAAACTCTACCAGGAGCCCAGAGAGCT
GCCTCCTGAGGAGAGAGCCCAGAATGTCACTCGGCTTCTACGAAAGCAGGCTGAGG
AGGTCAATACTGAGTGGGAAAAATTGAACCTGCACTCCGCTGACTGGCAGAGAAAA
ATAGATGAGACCCTTGAAAGACTCCAGGAACTTCAAGAGGCCACGGATGAGCTGGA
CCTCAAGCTGCGCCAAGCTGAGGTGATCAAGGGATCCTGGCAGCCCGTGGGCGATC
TCCTCATTGACTCTCTCCAAGATCACCTCGAGAAAGTCAAGGCACTTCGAGGAGAA
ATTGCGCCTCTGAAAGAGAACGTGAGCCACGTCAATGACCTTGCTCGCCAGCTTACC
ACTTTGGGCATTCAGCTCTCACCGTATAACCTCAGCACTCTGGAAGACCTGAACACC
AGATGGAAGCTTCTGCAGGTGGCCGTCGAGGACCGAGTCAGGCAGCTGCATGAAGC
CCACAGGGACTTTGGTCCAGCATCTCAGCACTTTCTTTCCACGTCTGTCCAGGGTCC
CTGGGAGAGAGCCATCTCGCCAAACAAAGTGCCCTACTATATCAACCACGAGACTC
AAACAACTTGCTGGGACCATCCCAAAATGACAGAGCTCTACCAGTCTTTAGCTGACC
TGAATAATGTCAGATTCTCAGCTTATAGGACTGCCATGAAACTCCGAAGACTGCAG
AAGGCCCTTTGCTTGGATCTCTTGAGCCTGTCAGCTGCATGTGATGCCTTGGACCAG
CACAACCTCAAGCAAAATGACCAGCCCATGGATATCCTGCAGATTATTAATTGTTTG
ACCACTATTTATGACCGCCTGGAGCAAGAGCACAACAATTTGGTCAACGTCCCTCTC
TGCGTGGATATGTGTCTGAACTGGCTGCTGAATGTTTATGATACGGGACGAACAGG
GAGGATCCGTGTCCTGTCTTTTAAAACTGGCATCATTTCCCTGTGTAAAGCACATTT
GGAAGACAAGTACAGATACCTTTTCAAGCAAGTGGCAAGTTCAACAGGATTTTGTG
ACCAGCGCAGGCTGGGCCTCCTTCTGCATGATTCTATCCAAATTCCAAGACAGTTGG
GTGAAGTTGCATCCTTTGGGGGCAGTAACATTGAGCCAAGTGTCCGGAGCTGCTTCC
AATTTGCTAATAATAAGCCAGAGATCGAAGCGGCCCTCTTCCTAGACTGGATGAGA
CTGGAACCCCAGTCCATGGTGTGGCTGCCCGTCCTGCACAGAGTGGCTGCTGCAGA
AACTGCCAAGCATCAGGCCAAATGTAACATCTGCAAAGAGTGTCCAATCATTGGAT
TCAGGTACAGGAGTCTAAAGCACTTTAATTATGACATCTGCCAAAGCTGCTTTTTTT
CTGGTCGAGTTGCAAAAGGCCATAAAATGCACTATCCCATGGTGGAATATTGCACTC
CGACTACATCAGGAGAAGATGTTCGAGACTTTGCCAAGGTACTAAAAAACAAATTT
CGAACCAAAAGGTATTTTGCGAAGCATCCCCGAATGGGCTACCTGCCAGTGCAGAC
TGTCTTAGAGGGGGACAACATGGAAACTGACACAATG
SEQ ID NO: 9 (POLYADENYLATION SIGNAL SEQUENCE)
AATAAAAGATCCTTATTTTCATTGGATCTGTGTGTTGGTTTTTTGTGT
SEQ ID NO: 10 (MD5 MICRO-DYSTROPHIN PROTEIN)
MLWWEEVEDCYEREDVQKKTFTKWVNAQFSKFGKQHIENLFSDLQDGRRLLDLLEGL
TGQKLPKEKGSTRVHALNNVNKALRVLQNNNVDLVNIGSTDIVDGNHKLTLGLIWNIIL
HWQVKNVMKNIMAGLQQTNSEKILLSWVRQSTRNYPQVNVINFTTSWSDGLALNALIH
SHRPDLFDWNSVVCQQSATQRLEHAFNIARYQLGIEKLLDPEDVDTTYPDKKSILMYITS
LFQVLPQQVSIEAIQEVEMLPRPPKVTKEEHFQLHHQMHYSQQITVSLAQGYERTSSPKP
RFKSYAYTQAAYVTTSDPTRSPFPSQHLEAPEDKSFGSSLMESEVNLDRYQTALEEVLS
WLLSAEDTLQAQGEISNDVEVVKDQFHTHEGYMMDLTAHQGRVGNILQLGSKLIGTGK
LSEDEETEVQEQMNLLNSRWECLRVASMEKQSNLHSYVPSTYLTEITHVSQALLEVEQL
LNAPDLCAKDFEDLFKQEESLKNIKDSLQQSSGRIDIIHSKKTAALQSATPVERVKLQEA
LSQLDFQWEKVNKMYKDRQGRFDRSVEKWRRFHYDIKIFNQWLTEAEQFLRKTQIPEN
WEHAKYKWYLKELQDGIGQRQTVVRTLNATGEEIIQQSSKTDASILQEKLGSLNLRWQ
EVCKQLSDRKKRLEEQSDQWKRLHLSLQELLVWLQLKDDELSRQAPIGGDFPAVQKQN
DVHRAFKRELKTKEPVIMSTLETVRIFLTEQPLEGLEKLYQEPRELPPEERAQNVTRLLR
KQAEEVNTEWEKLNLHSADWQRKIDETLERLQELQEATDELDLKLRQAEVIKGSWQPV
GDLLIDSLQDHLEKVKALRGEIAPLKENVSHVNDLARQLTTLGIQLSPYNLSTLEDLNTR
WKLLQVAVEDRVRQLHEAHRDFGPASQHELSTSVQGPWERAISPNKVPYYINHETQTT
CWDHPKMTELYQSLADLNNVRFSAYRTAMKLRRLQKALCLDLLSLSAACDALDQHNL
KQNDQPMDILQINCLTTIYDRLEQEHNNLVNVPLCVDMCLNWLLNVYDTGRTGRIRVL
SFKTGIISLCKAHLEDKYRYLFKQVASSTGFCDQRRLGLLLHDSIQIPRQLGEVASFGGSN
IEPSVRSCFQFANNKPEIEAALFLDWMRLEPQSMVWLPVLHRVAAAETAKHQAKCNIC
KECPIIGFRYRSLKHFNYDICQSCFFSGRVAKGHKMHYPMVEYCTPTTSGEDVRDFAKV
LKNKFRTKRYFAKHPRMGYLPVQTVLEGDNMETDTM
SEQ ID NO: 11 (AAVhu37 Nucleic Acid Sequence)
ATGGCTGCTGACGGTTATCTTCCAGATTGGCTCGAGGACAACCTCTCTGAGGGCATT
CGCGAGTGGTGGGACCTGAAACCTGGAGCCCCCAAGCCCAAGGCCAACCAGCAGA
AGCAGGACGACGGCCGGGGTCTGGTGCTTCCTGGCTACAAGTACCTCGGACCCTTC
AACGGACTCGACAAGGGGGAGCCCGTCAACGCGGCGGACGCAGCGGCCCTCGAGC
ACGACAAGGCCTACGACCAGCAGCTCAAAGCGGGTGACAATCCGTACCTGCGGTAT
AACCACGCCGACGCCGAGTTTCAGGAGCGTCTGCAAGAAGATACGTCTTTTGGGGG
CAACCTCGGGCGAGCAGTCTTCCAGGCCAAGAAGCGGGTTCTCGAACCTCTCGGTCT
GGTTGAGGAAGCTGCTAAGACGGCTCCTGGAAAGAAGAGACCGGTAGAACCGTCAC
CTCAGCGTTCCCCCGACTCCTCCACGGGCATCGGCAAGAAAGGCCAGCAGCCCGCT
AAAAAGAGACTGAACTTTGGTCAGACTGGCGACTCAGAGTCAGTCCCCGACCCTCA
ACCAATCGGAGAACCACCAGCAGGCCCCTCTGGTCTGGGATCTGGTACAATGGCTG
CAGGCGGTGGCGCTCCAATGGCAGACAATAACGAAGGCGCCGACGGAGTGGGTAG
TTCCTCAGGAAATTGGCATTGCGATTCCACATGGCTGGGCGACAGAGTCATCACCAC
CAGCACCCGAACCTGGGCCCTGCCCACCTACAACAACCACCTCTACAAGCAAATAT
CCAATGGGACATCGGGAGGAAGCACCAACGACAACACCTACTTCGGCTACAGCACC
CCCTGGGGGTATTTTGACTTCAACAGATTCCACTGCCACTTCTCACCACGTGACTGG
CAGCGACTCATCAACAACAACTGGGGATTCCGGCCAAAAAGACTCAGCTTCAAGCT
CTTCAACATCCAGGTCAAGGAGGTCACGCAGAATGAAGGCACCAAGACCATCGCCA
ATAACCTTACCAGCACGATTCAGGTATTTACGGACTCGGAATACCAGCTGCCGTACG
TCCTCGGCTCCGCGCACCAGGGCTGCCTGCCTCCGTTCCCGGCGGACGTCTTCATGA
TTCCCCAGTACGGCTACCTTACACTGAACAATGGAAGTCAAGCCGTAGGCCGTTCCT
CCTTCTACTGCCTGGAATATTTTCCATCTCAAATGCTGCGAACTGGAAACAATTTTG
AATTCAGCTACACCTTCGAGGACGTGCCTTTCCACAGCAGCTACGCACACAGCCAG
AGCTTGGACCGACTGATGAATCCTCTCATCGACCAGTACCTGTACTACTTATCCAGA
ACTCAGTCCACAGGAGGAACTCAAGGTACCCAGCAATTGTTATTTTCTCAAGCTGGG
CCTGCAAACATGTCGGCTCAGGCTAAGAACTGGCTACCTGGACCTTGCTACCGGCA
GCAGCGAGTCTCTACGACACTGTCGCAAAACAACAACAGCAACTTTGCTTGGACTG
GTGCCACCAAATATCACCTGAACGGAAGAGACTCTTTGGTAAATCCCGGTGTCGCC
ATGGCAACCCACAAGGACGACGAGGAACGCTTCTTCCCGTCGAGTGGAGTCCTGAT
GTTCGGAAAACAGGGTGCTGGAAGAGACAATGTGGACTACAGCAGCGTTATGCTAA
CCAGCGAAGAAGAAATTAAAACCACTAACCCCGTAGCCACAGAACAATACGGTGTG
GTGGCTGACAACTTGCAGCAAACCAATACAGGGCCTATTGTGGGAAATGTCAACAG
CCAAGGAGCCTTACCTGGCATGGTCTGGCAGAACCGAGACGTGTACCTGCAGGGTC
CCATCTGGGCCAAGATTCCTCACACGGACGGCAACTTCCACCCTTCACCGCTAATGG
GAGGATTTGGACTGAAGCACCCACCTCCTCAGATCCTGATCAAGAACACGCCGGTA
CCTGCGGATCCTCCAACAACGTTCAGCCAGGCGAAATTGGCTTCCTTCATTACGCAG
TACAGCACCGGACAGGTCAGCGTGGAAATCGAGTGGGAGCTGCAGAAGGAGAACA
GCAAACGCTGGAACCCAGAGATTCAGTACACTTCAAACTACTACAAATCTACAAAT
GTGGACTTTGCTGTCAATACAGAGGGAACTTATTCTGAGCCTCGCCCCATTGGTACT
CGTTACCTCACCCGTAATCTGTAA
SEQ ID NO: 12 (AAVhu37 Amino Acid Sequence)
MAADGYLPDWLEDNLSEGIREWWDLKPGAPKPKANQQKQDDGRGLVLPGYKYLGPF
NGLDKGEPVNAADAAALEHDKAYDQQLKAGDNPYLRYNHADAEFQERLQEDTSFGG
NLGRAVFQAKKRVLEPLGLVEEAAKTAPGKKRPVEPSPQRSPDSSTGIGKKGQQPAKKR
LNFGQTGDSESVPDPQPIGEPPAGPSGLGSGTMAAGGGAPMADNNEGADGVGSSSGNW
HCDSTWLGDRVITTSTRTWALPTYNNHLYKQISNGTSGGSTNDNTYFGYSTPWGYFDF
NRFHCHFSPRDWQRLINNNWGFRPKRLSFKLFNIQVKEVTQNEGTKTIANNLTSTIQVFT
DSEYQLPYVLGSAHQGCLPPFPADVFMIPQYGYLTLNNGSQAVGRSSFYCLEYFPSQML
RTGNNFEFSYTFEDVPFHSSYAHSQSLDRLMNPLIDQYLYYLSRTQSTGGTQGTQQLLFS
QAGPANMSAQAKNWLPGPCYRQQRVSTTLSQNNNSNFAWTGATKYHLNGRDSLVNP
GVAMATHKDDEERFFPSSGVLMFGKQGAGRDNVDYSSVMLTSEEEIKTTNPVATEQYG
VVADNLQQTNTGPIVGNVNSQGALPGMVWQNRDVYLQGPIWAKIPHTDGNFHPSPLM
GGFGLKHPPPQILIKNTPVPADPPTTFSQAKLASFITQYSTGQVSVEIEWELQKENSKRW
NPEIQYTSNYYKSTNVDFAVNTEGTYSEPRPIGTRYLTRNL
SEQ ID NO: 13 (Streptococcus pyogenes IdeS)
DSFSANQEIRYSEVTPYHVTSVWTKGVTPPANFTQGEDVFHAPYVANQGWYDITKTEN
GKDDLLCGAATAGNMLHWWFDQNKDQIKRYLEEHPEKQKINFNGEQMFDVKEAIDTK
NHQLDSKLFEYFKEKAFPYLSTKHLGVFPDHVIDMFINGYRLSLTNHGPTPVKEGSKDP
RGGIFDAVFTRGDQSKLLTSRHDFKEKNLKEISDLIKKELTEGKALGLSHTYANVRINHV
INLWGADFDSNGNLKAIYVTDSDSNASIGMKKYFVGVNSAGKVAISAKEIKEDNIGAQV
LGLFTLSTGQDSWNQTN
SEQ ID NO: 14 (Streptococcus equi IdeZ)
MKTIAYPNKPHSLSAGLLTAIAIFSLASSNITYADDYQRNATEAYAKEVPHQITSVWSKG
VTPLTPEQFRYNNEDVIHAPYLAHQGWYDITKAFDGKDNLLCGAATAGNMLHWWFDQ
NKTEIEAYLSKHPEKQKIIFNNQELFDLKAAIDTKDSQTNSQLFNYFRDKAFPNLSARQL
GVMPDLVLDMFINGYYLNVFKTQSTDVNRPYQDKDKRGGIFDAVFTRGDQTTLLTARH
DLKNKGLNDISTIIKQELTEGRALALSHTYANVSISHVINLWGADFNAEGNLEAIYVTDS
DANASIGMKKYFVGINAHGHVAISAKKIEGENIGAQVLGLFTLSSGKDIWQKLS

What is claimed is:

1. A recombinant adeno-associated virus (rAAV), wherein said rAAV comprises an AAV capsid and a vector genome packaged therein, wherein said vector genome comprises a nucleic acid sequence that is at least 99% identical to SEQ ID NO: 1.

2. The rAAV according to claim 1, wherein the nucleic acid sequence comprises the sequence set forth in SEQ ID NO: 1.

3. The rAAV according to claim 1, wherein the AAV capsid is from an AAV of serotype hu37, 8, 1, 2, 3, 4, 5, 6, 7, 9, 10, 11, 12, rh10, or rh74.

4. The rAAV according to claim 3, wherein the AAV capsid is an hu37 capsid.

5. The rAAV according to claim 3, wherein the AAV capsid is an AAV8 capsid.

6. The rAAV according to claim 3, wherein the AAV capsid is an AAV9 capsid.

7. The rAAV according to any one of the preceding claims, wherein the packaged vector genome further comprises a muscle specific control element.

8. The rAAV according to claim 7, wherein the muscle specific control element is selected from a CK8 promoter, a CK7 promoter, a CK9 promoter, a muscle specific creatine kinase (MCK) promoter, truncated MCK (tMCK), myosin heavy chain (MHC), a hybrid α-myosin heavy chain enhancer-/MCK (MHCK7) enhancer-promoter, a human skeletal actin gene element, a cardiac actin gene element, a myocyte-specific enhancer binding factor mef, C5-12, a murine creatine kinase enhancer element, a skeletal fast-twitch troponin c gene element, a slow-twitch cardiac troponin c gene element, the slow-twitch troponin i gene element, a hypoxia-inducible nuclear factor, a steroid-inducible element, and a glucocorticoid response element.

9. The rAAV according to claim 8, wherein the muscle specific control element is a CK8 promoter.

10. The rAAV according to claim 9, wherein the CK8 promoter comprises the sequence set forth in SEQ ID NO: 6.

11. The rAAV according to claim 8, wherein the muscle specific control element is a hybrid α-myosin heavy chain enhancer-/MCK (MHCK7) enhancer-promoter.

12. The rAAV according to claim 11, wherein the MHCK7 enhancer-promoter comprises the sequence set forth in SEQ ID NO: 7.

13. The rAAV according to any one of the preceding claims, wherein the packaged vector genome further comprises a 5′-ITR sequence.

14. The rAAV according to any one of the preceding claims, wherein the packaged vector genome further comprises a 3′-ITR sequence.

15. The rAAV according to claim 13 or 14, wherein the 5′-ITR sequence and/or the 3′-ITR sequence are from AAV2.

16. The rAAV according to claim 13 or 14, wherein the 5′-ITR sequence and/or the 3′-ITR sequence are from a non-AAV2 source.

17. The rAAV according to any one of the preceding claims, wherein the packaged vector genome further comprises one or more intron sequences.

18. The rAAV according to claim 17, wherein the intron is selected from a SV40 Small T intron, a rabbit hemoglobin subunit beta (rHBB) intron, a human β-globin/IgG chimeric intron, a human beta globin IVS2 intron, and an hFIX intron.

19. The rAAV according to any one of the preceding claims, wherein the packaged vector genome further comprises a polyadenylation signal sequence.

20. The rAAV according to claim 19, wherein the polyadenylation signal sequence is selected from a synthetic polyadenylation signal sequence, an SV40 polyadenylation signal sequence, a bovine growth hormone (BGH) polyadenylation signal sequence, and a rabbit beta globin polyadenylation signal sequence.

21. The rAAV according to claim 20, wherein the polyadenylation signal sequence is a synthetic polyadenylation signal sequence.

22. The rAAV according to claim 21, wherein the synthetic polyadenylation signal sequence comprises the sequence set forth in SEQ ID NO: 9.

23. The rAAV according to claim 1, wherein the packaged vector genome comprises the sequence set forth in SEQ ID NO: 3.

24. A recombinant adeno-associated virus (rAAV), wherein said rAAV comprises an AAVhu37 capsid and a vector genome packaged therein, wherein said vector genome comprises a nucleic acid sequence encoding the MD5 micro-dystrophin polypeptide set forth in SEQ ID NO: 10.

25. The rAAV according to claim 24, wherein the nucleic acid sequence encoding the MD5 micro-dystrophin polypeptide comprises a sequence that is at least 99% identical to SEQ ID NO:
1.

26. The rAAV according to claim 24, wherein the nucleic acid sequence encoding the MD5 micro-dystrophin polypeptide comprises the sequence set forth in SEQ ID NO: 1.

27. The rAAV according to any one of claims 24-26, wherein the packaged vector genome further comprises a muscle specific control element.

28. The rAAV according to claim 27, wherein the muscle specific control element is selected from a CK8 promoter, a CK7 promoter, a CK9 promoter, a muscle specific creatine kinase (MCK) promoter, truncated MCK (tMCK), myosin heavy chain (MHC), a hybrid α-myosin heavy chain enhancer-/MCK (MHCK7) enhancer-promoter, a human skeletal actin gene element, a cardiac actin gene element, a myocyte-specific enhancer binding factor mef, C5-12, a murine creatine kinase enhancer element, a skeletal fast-twitch troponin c gene element, a slow-twitch cardiac troponin c gene element, the slow-twitch troponin i gene element, a hypoxia-inducible nuclear factor, a steroid-inducible element, and a glucocorticoid response element.

29. The rAAV according to claim 28, wherein the muscle specific control element is a CK8 promoter.

30. The rAAV according to claim 29, wherein the CK8 promoter comprises the sequence set forth in SEQ ID NO: 6.

31. The rAAV according to claim 28, wherein the muscle specific control element is a hybrid α-myosin heavy chain enhancer-/MCK (MHCK7) enhancer-promoter.

32. The rAAV according to claim 31, wherein the MHCK7 enhancer-promoter comprises the sequence set forth in SEQ ID NO: 7.

33. The rAAV according to any one of claims 24-32, wherein the packaged vector genome further comprises a 5′-ITR sequence.

34. The rAAV according to any one of claims 24-33, wherein the packaged vector genome further comprises a 3′-ITR sequence.

35. The rAAV according to claim 33 or 34, wherein the 5′-ITR sequence and/or the 3′-ITR sequence are from AAV2.

36. The rAAV according to claim 33 or 34, wherein the 5′-ITR sequence and/or the 3′-ITR sequence are from a non-AAV2 source.

37. The rAAV according to any one of claims 24-36, wherein the packaged vector genome further comprises one or more intron sequences.

38. The rAAV according to claim 37, wherein the intron is selected from a SV40 Small T intron, a rabbit hemoglobin subunit beta (rHBB) intron, a human β-globin/IgG chimeric intron, a human beta globin IVS2 intron, and an hFIX intron.

39. The rAAV according to any one of claims 24-38, wherein the packaged vector genome further comprises a polyadenylation signal sequence.

40. The rAAV according to claim 39, wherein the polyadenylation signal sequence is selected from a synthetic polyadenylation signal sequence, an SV40 polyadenylation signal sequence, a bovine growth hormone (BGH) polyadenylation signal sequence, and a rabbit beta globin polyadenylation signal sequence.

41. The rAAV according to claim 40, wherein the polyadenylation signal sequence is a synthetic polyadenylation signal sequence.

42. The rAAV according to claim 41, wherein the synthetic polyadenylation signal sequence comprises the sequence set forth in SEQ ID NO: 9.

43. The rAAV according to claim 24, wherein the packaged vector genome comprises the sequence set forth in SEQ ID NO: 3.

44. A composition comprising the rAAV of any one of the preceding claims and a pharmaceutically acceptable carrier.

45. A method of treating a muscular dystrophy in a human subject comprising administering to the human subject a therapeutically effective amount of an rAAV of any one of claims 1-43 or a composition of claim 44.

46. A method of treating a muscular dystrophy in a human subject comprising first administering to the human subject an IgG-degrading protease and then subsequently administering a therapeutically effective amount of an rAAV of any one of claims 1-43 or a composition of claim 44.

47. A method of treating a muscular dystrophy in a human subject comprising administering a therapeutically effective amount of an rAAV of any one of claims 1-43 or a composition of claim 44, wherein the human subject has been administered an IgG-degrading protease.

48. The method of claim 46 or 47, wherein the IgG-degrading protease is IdeS of Streptococcus pyogenes or an engineered variant thereof.

49. The method of claim 46 or 47, wherein the IgG-degrading protease is IdeZ of Streptococcus equi or an engineered variant thereof.

50. The method according to any one of claims 45-49, wherein the muscular dystrophy is caused by a mutation in the dystrophin gene.

51. The method of claim 50, wherein the muscular dystrophy is selected from Duchenne muscular dystrophy (DMD), Becker muscular dystrophy, and X-linked dilated cardiomyopathy.

52. The method according to any one of claims 45-51, wherein the rAAV or the composition is administered intravenously, subcutaneously, intramuscularly, intradermally, intraperitoneally, or intrathecally.

53. The method of claim 52, wherein the rAAV or the composition is administered intravenously.

54. The method of claim 52, wherein the rAAV or the composition is administered intramuscularly.

55. The method according to any one of claims 45-54, wherein the rAAV is administered at a dose of about 1×10¹² genome copies (GC)/kg to about 1×10¹⁶ genome copies (GC)/kg.

56. The method of claim 55, wherein the rAAV is administered at a dose of about 1×10¹³ genome copies (GC)/kg to about 1×10¹⁵ genome copies (GC)/kg.

57. The method of claim 56, wherein the rAAV is administered at a dose of about 1×10¹⁴ genome copies (GC)/kg.

58. A polynucleotide which comprises a nucleic acid sequence at least 99% identical to the sequence of SEQ ID NO: 1.

59. A polynucleotide which comprises the nucleic acid sequence set forth in SEQ ID NO: 1.

60. A polynucleotide which consists of the nucleic acid sequence set forth in SEQ ID NO: 1.

61. A polynucleotide which comprises a nucleic acid sequence at least 99% identical to the sequence of SEQ ID NO: 3.

62. A polynucleotide which comprises the nucleic acid sequence set forth in SEQ ID NO: 3.

63. A polynucleotide which consists of the nucleic acid sequence set forth in SEQ ID NO: 3.

64. A host cell comprising a polynucleotide of any one of claims 58-63.

65. The host cell of claim 64, wherein the host cell is selected from a HeLa cell, a Cos-7 cell, a HEK293 cell, an A549 cell, a BHK cell, a Vero cell, an RD cell, an HT-1080 cell, an ARPE-19 cell, and a MRC-5 cell.

66. The host cell of claim 65, wherein the host cell is a HeLa cell.

67. The host cell of claim 66, wherein the HeLa cell has been engineered to inactivate one or more endogenous genes.

68. The host cell of claim 67, wherein the endogenous gene is selected from KCNN2, RGMA, ATP5EP2, LINC00319, CYP3A7, ABCA10, NOG, SPANXN3, PGAS, MYRIP, and NALCN-AS1.

69. The host cell of claim 68, wherein the endogenous gene is RGMA.

70. The host cell of claim 68, wherein the endogenous gene is KCNN2.
